(19) United States
(12) Patent Application Publication
(10) Pub. No.: US 2024/0002905 A1
(43) Pub. Date: Jan. 4, 2024

(54) PATHOGEN INACTIVATING AND NUCLEIC ACID STABILIZATION MEDIA FOR MICROORGANISM COLLECTION AND TRANSPORT

(71) Applicants: Lyle ARNOLD, San Diego, CA (US); Biocept, Inc., San Diego, CA (US)

(72) Inventor: Lyle ARNOLD, Poway, CA (US)

(73) Assignee: Biocept, Inc., San Diego, CA (US)

(21) Appl. No.: 18/252,684

(22) PCT Filed: Nov. 11, 2021
(86) PCT No.: PCT/US2021/059004

§ 371 Date: May 11, 2023

Related U.S. Application Data
(60) Provisional application No. 63/112,384, filed on Nov. 11, 2020.

(51) Int. Cl. C12Q 1/6806; C12Q 1/70; C12Q 1/02

(52) U.S. Cl. CPC C12Q 1/6806; C12Q 1/701; C12Q 1/02

(57) ABSTRACT

The present disclosure provides pathogen inactivating formulations, and methods of use thereof in collecting, transporting and storing biological samples, as well as preventing degradation of and purifying nucleic acids. The disclosure further provides methods of preparing compositions comprising the pathogen inactivating formulation and a biological sample.

### CROSS REFERENCE TO RELATED APPLICATIONS

This application claims the benefit of U.S. Provisional Application No. 63/112,384 filed on Nov. 11, 2020, the contents of which are herein incorporated by reference in its entirety for all purposes.

### BACKGROUND

Diagnosis of infections or diseases in human subjects is often based on results from diagnostic assays conducted on a biological sample obtained from the subject or the environment. In some cases, the diagnostic assays are designed to detect the presence of a pathogenic microbe, or a nucleic acid or protein derived from the microbe. For diagnostic assays to work effectively and reliably, it is important to be able to collect, transport and store the biological sample in a way that preserves the integrity of nucleic acids, proteins and other molecules in the biological sample. Nucleic acids, especially ribonucleic acid (RNA), are particularly susceptible to degradation at room temperature and must generally be stored under freezing temperatures to remain stable.

The transportation and storage of biological samples assume further importance when there is considerable time lag between collection of the sample and performance of the diagnostic assay at a laboratory. Additionally, it is important to take adequate measures to ensure the safety of personnel who handle biological samples containing or suspected to contain pathogenic microbes. Such safety measures can be cumbersome and expensive. Additionally, formulations that are typically used to collect, transport and store biological samples often have undesirable properties, such as being incompatible with the use of bleach in the vicinity of the formulations. Therefore, there is an urgent need for formulations and methods that may be used to collect, transport and store biological samples in a safe and effective manner, particularly in view of the ongoing COVID-19 global pandemic.

### SUMMARY

The disclosure provides formulations, comprising a sodium salt of a detergent, a metal chelating agent and a buffer, wherein the concentration of the detergent is in the range of about 0.1% to about 3%, wherein the concentration of the metal chelating agent is in the range of about 0.5 mM to about 1.5 mM, and wherein the concentration of the buffer is in the range of about 20 mM to about 40 mM. In some embodiments, the contact of the formulation with a biological sample results in one or more of the following: (a) inactivation of a nuclease in the biological sample, (b) inactivation of a pathogenic microbe in the biological sample, (c) disruption of an association of a protein with a nucleic acid in the biological sample, and (d) reduction or prevention of degradation of a nucleic acid in the biological sample, wherein the biological sample is derived from a subject, or the environment.

The disclosure provides formulations, comprising the following components: a sodium salt of a detergent, a metal chelating agent and a buffer, wherein contact of the formulation with a biological sample results in one or more of the following: (a) inactivation of a nuclease in the biological sample, (b) inactivation of a pathogenic microbe in the biological sample, (c) disruption of an association of a protein with a nucleic acid in the biological sample, and (d) reduction or prevention of degradation of a nucleic acid in the biological sample, wherein the biological sample is derived from a subject.

In some embodiments, the concentration of the detergent is in the range of about 1% to about 3%. In some embodiments, the concentration of the detergent is about 2%. In some embodiments, the concentration of the detergent is about 3%. In some embodiments, the detergent is selected from the group consisting of sodium lauryl sulfate, sodium lauryl sarcosine, sodium octyl sulfate, sodium dihexadecyl phosphate, sodium taurodeoxycholate, sodium taurocholate, sodium glycocholate, sodium deoxycholate, sodium cholate, and sodium alkylbenzene sulfonate. In some embodiments, the detergent is sodium lauryl sulfate.

In some embodiments, the metal chelating agent is selected from the group consisting of ethylene glycol tetraacetic acid (EGTA), hydroxyl ethylethylenediaminetriacetic acid, diethylene triamine penta acetic acid, N,N-bis(carboxymethyl)glycine, ethylene diamine tetraacetic acid (EDTA), citrate anhydrous, sodium citrate, calcium citrate, ammonium citrate, ammonium bicitrate, citric acid, diammonium citrate, ferric ammonium citrate, and lithium citrate. In some embodiments, the metal chelating agent is EDTA, EGTA, or a combination thereof. In some embodiments, the concentration of the metal chelating agent is about 1 mM.

In some embodiments, the buffer comprises tris(hydroxymethyl)aminomethane, citrate, 2-(N-morpholino)ethanesulfonic acid, N,N-Bis(2-hydroxyethyl)-2-aminoethanesulfonic acid, 1,3-bis(tris(hydroxymethyl)methyl amino)propane, 4-(2-hydroxy ethyl)-1-piperazine ethanesulfonic acid, 3-(N-morpholino) propanesulfonic acid, bicarbonate, or phosphate. In some embodiments, the buffer comprises a phosphate buffer. In some embodiments, the concentration of the buffer is about 30 mM. In some embodiments, the formulation has a pH in the range of about 6.5 to about 7.5. In some embodiments, the formulation has a pH of about 6.7.

In some embodiments, the formulation does not comprise a chaotropic agent. In some embodiments, the chaotropic agent is selected from the group consisting of n-butanol, ethanol, guanidine isocyanate, guanidine hydrochloride, guanidinium thiocyanate, guanidinium chloride, lithium perchlorate, lithium acetate, magnesium chloride, phenol, 2-propanol, thiourea, and urea. In some embodiments, the chaotropic agent is guanidinium thiocyanate. In some embodiments, the formulation does not comprise a reducing agent. In some embodiments, the reducing agent is selected from the group consisting of 2-mercaptoethanol, tris(2-carboxyethyl)phosphine, dithiothreitol, dim ethyl sulfoxide, and tris(2-carboxyethyl)phosphine. In some embodiments, bringing the formulation in contact with bleach does not produce a hazardous compound. In some embodiments, the hazardous compound is hydrogen cyanide.

In some embodiments, the biological sample comprises a nasal sample, an oral sample, a saliva sample, a urine sample, a stool sample, a bronchoalveolar lavage sample, a nasopharyngeal aspirate sample or a blood sample. In some embodiments, the biological sample comprises a nasal sample, an oral sample, a bronchoalveolar lavage sample, a nasopharyngeal aspirate sample or a saliva sample. In some embodiments, the biological sample is obtained from a nasopharyngeal swab, an oropharyngeal swab, or a combination thereof.

In some embodiments, contact of the formulation with the biological sample results in inactivation of a nuclease in the biological sample. In some embodiments, the nuclease is an RNase. In some embodiments, the biological sample comprises, or is suspected to comprise a nucleic acid derived from a pathogenic microbe. In some embodiments, the biological sample comprises, or is suspected to comprise a pathogenic microbe. In some embodiments, the subject has or is at a risk of developing a disease associated with, correlated with, or caused by a pathogenic microbe. In some embodiments, contact of the formulation with the biological sample results in inactivation of a pathogenic microbe in the biological sample. In some embodiments, the pathogenic microbe is capable of causing disease in a healthy subject. In some embodiments, the inactivated pathogenic microbe is incapable of causing disease in the healthy subject.

In some embodiments, the pathogenic microbe is a bacterium, fungus, virus, or parasite. In some embodiments, the pathogenic microbe is a virus. In some embodiments, the virus is selected from the group consisting of SARS-CoV-2, SARS-CoV-1, MERS-CoV, chikungunya virus, African Swine Fever virus, Dengue virus, Zika virus, Influenza virus A, Influenza virus B, Influenza virus C, Human Immunodeficiency Virus (HIV), Ebola virus, Hepatitis virus A, Hepatitis virus B, Hepatitis virus C, Hepatitis virus D, and Hepatitis virus E, herpes simplex virus type 1 (HSV-1), herpes simplex virus type 2 (HSV-2) and Human Papillomavirus. In some embodiments, the virus is SARS-CoV2.

In some embodiments, contact of the formulation with the biological sample results in disruption of an association of a protein with a nucleic acid in the biological sample. In some embodiments, the association of the protein with the nucleic acid comprises a physical binding of the protein with the nucleic acid. In some embodiments, contact of the formulation with a biological sample results in reduction or prevention of degradation of a nucleic acid in the biological sample. In some embodiments, degradation of the nucleic acid is reduced or prevented for at least about 1 day. In some embodiments, degradation of the nucleic acid is reduced or prevented for at least about 1 day at room temperature. In some embodiments, degradation of the nucleic acid is reduced or prevented for at least about 3 days at room temperature. In some embodiments, degradation of the nucleic acid is reduced or prevented for at least about 1 month at room temperature.

In some embodiments, the nucleic acid is derived from a pathogenic microbe. In some embodiments, the nucleic acid is an RNA, a DNA, or a combination thereof. In some embodiments, the nucleic acid is an RNA. In some embodiments, the RNA is a SARS-CoV-2 genomic RNA.

The disclosure provides formulations, comprising: (a) about 3% sodium lauryl sulfate; (b) about 1 mM each of the EDTA and EGTA; and (c) about 30 mM of phosphate buffer; wherein the formulation has a pH of about 6.7, and wherein the formulation does not comprise guanidinium thiocyanate. The disclosure further provides containers comprising any of the formulations disclosed herein. The disclosure further provides sample collection systems, comprising: a collection device or a collection vessel; and any of the formulations disclosed herein.

The disclosure provides methods of preparing a composition, comprising (a) collecting a biological sample from a subject, and (b) bringing the biological sample in contact with any of the formulations disclosed herein to form the composition. The disclosure also provides methods of transporting a biological sample derived from a subject, comprising (a) collecting the biological sample from the subject; (b) bringing the biological sample in contact with any of the formulations disclosed herein to form a composition; and (c) transporting the composition. In some embodiments, step (b) results in one or more of the following: (a) inactivation of a nuclease in the biological sample, (b) inactivation of a pathogenic microbe in the biological sample, (c) disruption of an association of a protein with a nucleic acid in the biological sample, and (d) reduction or prevention of degradation of a nucleic acid in the biological sample.

The disclosure also provides methods of inactivating a pathogenic microbe in a biological sample derived from a subject, comprising bringing the biological sample in contact with any of the formulations disclosed herein to form a composition. In some embodiments, the pathogenic microbe is capable of causing disease in a healthy subject. In some embodiments, the inactivated pathogenic microbe is incapable of causing disease in the healthy subject. In some embodiments, the pathogenic microbe is a bacterium, fungus, virus, or parasite. In some embodiments, the pathogenic microbe is a virus. In some embodiments, the virus is selected from the group consisting of SARS-CoV-2, SARS-CoV-1, MERS-CoV, chikungunya virus, African Swine Fever virus, Dengue virus, Zika virus, Influenza virus A, Influenza virus B, Influenza virus C, Human Immunodeficiency Virus (HIV), Ebola virus, Hepatitis virus A, Hepatitis virus B, Hepatitis virus C, Hepatitis virus D, and Hepatitis virus E, herpes simplex virus type 1 (HSV-1), herpes simplex virus type 2 (HSV-2) and Human Papillomavirus. In some embodiments, the virus is SARS-CoV2.

The disclosure provides methods of detecting a nucleic acid in a biological sample, comprising (a) collecting the biological sample from a subject, (b) bringing the biological sample in contact with at least one of the formulations disclosed herein to form a composition, and (c) detecting the nucleic acid in the composition. The disclosure provides methods of purifying a nucleic acid from a biological sample, comprising (a) collecting the biological sample from a subject, (b) bringing the biological sample in contact with at least one of the formulations disclosed herein to form a composition, and (c) purifying the nucleic acid from the composition.

In some embodiments, the method comprises purifying the nucleic acid. In some embodiments, purifying the nucleic acid comprises Boom extraction. In some embodiments, Boom extraction comprises bringing the nucleic acid in contact with silica. In some embodiments, purifying the nucleic acid comprises hybridization of the nucleic acid. In some embodiments, the nucleic acid is derived from a pathogenic microbe. In some embodiments, the nucleic acid is an RNA, a DNA, or a combination thereof. In some embodiments, the nucleic acid is an RNA. In some embodiments, the RNA is a SARS-CoV-2 genomic RNA.

In some embodiments, the method comprises transporting and/or storing the composition at room temperature. In some embodiments, the method comprises transporting and/or storing the composition at room temperature for at least 1 day. In some embodiments, the method further comprises transporting and/or storing the composition at room temperature for at least 1 month. In some embodiments, the method comprises amplifying the nucleic acid. In some embodiments, the method comprises amplifying the nucleic acid after storing the composition at room temperature for at least 1 day. In some embodiments, the method comprises amplifying the nucleic acid after storing the composition at room temperature for at least 3 days. In some embodiments, the method comprises amplifying the nucleic acid after storing the composition at room temperature for at least 1 month.

The disclosure provides kits comprising at least one of the formulations disclosed herein and/or at least one of the containers disclosed herein, and/or the at least one of the sample collection systems disclosed herein.

Further aspects and embodiments are provided by the detailed disclosure that follows. The invention is not limited by this summary.

### BRIEF DESCRIPTION OF FIGURES

FIG. 1 shows a graph showing the cycle threshold (CT) values obtained from RT-qPCR using primers against nucleoprotein N1 and N2 genes of the indicated samples containing different concentrations of SDS, which were stored at room temperature for 1 hour, 23 hours or 72 hours. See Example 1.

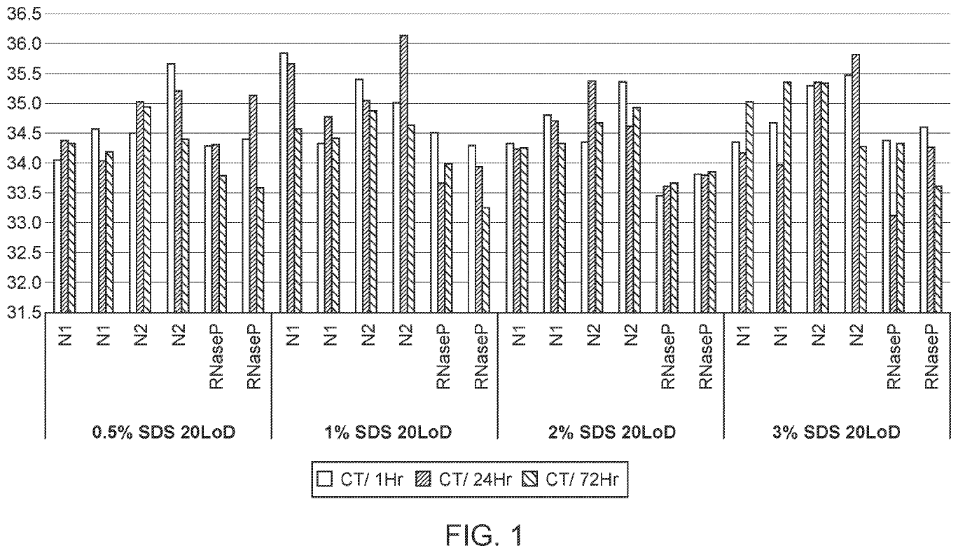

FIG. 2 shows the infectivity and toxicity following exposure to various dilutions of CoV229E stock samples, as indicated, to Formulation D for 2 hours. See Example 7.

### DETAILED DESCRIPTION

The disclosure provides pathogen inactivating formulations (interchangeably used with pathogen transport media formulations), and methods of use thereof in collecting, transporting and storing biological samples, preventing degradation of and purifying nucleic acids, and/or inactivating pathogens in the biological sample. The disclosure further provides methods of preparing compositions comprising at least one of the formulations disclosed herein and a biological sample.

### Definitions

The following terms are used in the description herein and the appended claims:

The singular forms “a,” “an” and “the” are intended to include the plural forms as well, unless the context clearly indicates otherwise.

Furthermore, the term “about” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant. In some embodiments, the subject is any animal host, including, but not limited to, human and non-human primates, avians, reptiles, amphibians, bovines, canines, caprines, cavines, corvines, epines, equines, felines, hircines, lapines, leporines, lupines, murines, ovines, por-cines, racines, vulpines, and the like, including, without limitation, domesticated livestock, herding or migratory animals or birds, exotics or zoological specimens, as well as companion animals, pets, and any animal under the care of a veterinary practitioner. The formulations and methods disclosed herein may also be used to monitor the outbreak, progression, and epidemiological statistics of a disease, such as, COVID-19, wasting disease in ungulates, tuberculosis, Ebola, SARS, and avian influenzas.

A “nucleic acid” or “polynucleotide” is a sequence of nucleotide bases, for example RNA, DNA or DNA-RNA hybrid sequences (including both naturally occurring and non-naturally occurring nucleotides). In some embodiments, the nucleic acids of the disclosure are either single or double stranded DNA sequences. A nucleic acid may be 1-1,000, 1,000-10,000, 10,000-100,000, 100,000-1 million or greater than 1 million nucleotides in length. A nucleic acid will generally contain phosphodiester bonds, although in some cases nucleic acid analogs are included that may have alternate backbones, comprising, for example, phosphoramide, phosphorothioate, phosphorodithioate, O-methylphophoroamidite linkages, and peptide nucleic acid backbones and linkages. Other analog nucleic acids include those with positive backbones, non-ionic backbones, and non-ribose backbones. Nucleic acids containing one or more carbocyclic sugars are also included within the definition of nucleic acids. These modifications of the ribose-phosphate backbone may facilitate the addition of labels, or to increase the stability and half-life of such molecules in physiological environments. Nucleic acids of the disclosure may be linear, or may be circular (e.g., a plasmid).

As used herein, a “biological sample” refers to a composition containing or presumed to contain a substance of interest, such as a composition of matter containing nucleic acid, protein, or another biomolecule of interest. In some embodiments, the biological sample is obtained from a subject. In some embodiments, the biological sample is obtained from the environment, such as, surfaces, soil, water and waste water. In some embodiments, the biological sample is used fresh, or has being stored for a period of time, including for example, cryopreserved samples and the like, and may include material of clinical, veterinary, environmental or forensic origin, may be isolated from food, beverages, feedstocks, potable water sources, wastewater streams, industrial waste or effluents, natural water sources, soil, airborne sources, pandemic or epidemic populations, epidemiological samples, research materials, pathology specimens, suspected bioterrorism agents, crime scene evidence, and the like.

The term “biological sample” may encompass a solution, cell, tissue, or population of one of more of the same that includes a population of nucleic acids (genomic DNA, cDNA, RNA, protein, other cellular molecules, etc.). Exemplary biological samples include, but are not limited to, whole blood, plasma, serum, sputum, urine, stool, white blood cells, red blood cells, buffy coat, swabs (including, without limitation, buccal swabs, throat swabs, vaginal swabs, urethral swabs, cervical swabs, rectal swabs, lesion swabs, abscess swabs, nasopharyngeal swabs, and the like), urine, stool, sputum, tears, mucus, saliva, semen, vaginal fluids, lymphatic fluid, amniotic fluid, spinal or cerebrospinal fluid, peritoneal effusions, pleural effusions, exudates, punctuates, epithelial smears, biopsies, bone marrow samples, fluid from cysts or abcess contents, synovial fluid, vitreous or aqueous humor, eye washes or aspirates, pulmonary lavage or lung aspirates, and organs and tissues, including but not limited to, liver, spleen, kidney, lung, intestine, brain, heart, muscle, pancreas, and the like, and any combination thereof. In some embodiments, the sample may be, or be from, an organism that acts as a vector, such as a mosquito, or tick, or other insect(s). Tissue culture cells, including explanted material, primary cells, secondary cell lines, and the like, as well as lysates, homogenates, extracts, or materials obtained from any cells, are also within the meaning of the term “biological sample” as used herein. In some embodiments, microorganisms (including, without limitation, prokaryotes such as the archaebacteria and eubacteria: cyanobacteria; fungi, yeasts, molds, actinomycetes; spirochetes, and mycoplasmas); viruses (including, without limitation the Orthohepadnaviruses including, e.g., hepatitis A, B, and C viruses, human papillomavirus, Flaviviruses including, e.g., Dengue virus. Lyssaviruses including, e.g., rabies virus. Morbilliviruses including, e.g., measles virus, Simplexviruses including, e.g., herpes simplex virus, Polyomaviruses, Rubulaviruses including, e.g., mumps virus, Rubiviruses including, e.g., rubella virus, Varicellovirus including, e.g., chickenpox virus, or rotavirus, coronavirus, cytomegalovirus, adenovirus, adeno-associated virus, baculovirus, parvovirus, retrovirus, vaccinia, poxvirus, and the like), algae, protozoans, protists, plants, bryophytes, and the like, and any combination of any of the foregoing, may be present on or in a biological sample. In some embodiments, biological sample refers to lysates, extracts, or materials obtained from any of the above exemplary biological samples.

The term “chaotrope” or “chaotropic agent” as used herein, refers to non-detergent substances that can increase disorder in a protein or nucleic acid by interfering with and/or weakening non-covalent forces, such as hydrogen bonds, van der Waals forces, and hydrophobic effects. In some embodiments, the chaotrope alters the secondary, tertiary, or quaternary structure of a protein or a nucleic acid while leaving the primary structure intact. In some embodiments, the chaotrope causes denaturation of the protein or nucleic acid.

The term “substantially free” or “essentially free,” as used herein, typically means that a composition contains less than about 10 weight percent, preferably less than about 5 weight percent, and more preferably less than about 1 weight percent of a compound. In a preferred embodiment, these terms refer to less than about 0.5 weight percent, more preferably less than about 0.1 weight percent or even less than about 0.01 weight percent. The terms encompass a composition being entirely free of a compound or other stated property, as well. With respect to degradation or deterioration, the term “substantial” may also refer to the above-noted weight percentages, such that preventing substantial degradation would refer to less than about 15 weight percent, less than about 10 weight percent, preferably less than about 5 weight percent, etc., being lost to degradation. In some embodiments, these terms refer to mere percentages rather than weight percentages, such as with respect to the term “substantially non-pathogenic” where the term “substantially” refers to leaving less than about 10 percent, less than about 5 percent, etc., of the pathogenic activity.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “COVID-19 disease” refers to a disease caused by the severe acute respiratory syndrome coronavirus 2 (SARS-CoV-2). SARS-CoV-2 was first discovered in Wuhan, China in December 2019. SARS-CoV-2 is an enveloped, non-segmented, positive-sense RNA virus. SARS-CoV-2 shares a high degree of amino acid and nucleic acid sequence similarity with SARS-CoV across its entire genome. In some embodiments, the SARS-CoV-2 is an S strain of SARS-CoV-2. In some embodiments, the SARS-CoV-2 is an L strain of SARS-CoV-2. In some embodiments, the SARS-CoV-2 is any strain of SARS-CoV-2 now known or later discovered.

As used herein, “inactivation” may refer to the process of rendering inactive a living organism, such as a microbe; or a molecule, such as a protein. In some embodiments, the inactivation of a protein that has enzymatic activity (such as, an enzyme) results in the loss of enzymatic activity. In some embodiments, the inactivation of a protein results in denaturation of the protein. In some embodiments, the inactivation of a microbe refers to the killing of the microbe. In some embodiments, the inactivation of a microbe refers to a reduction (attenuation) or loss of pathogenicity of the microbe.

### Formulations

The disclosure provides formulations, comprising the following components: a detergent, a metal chelating agent and a buffer. In some embodiments, the detergent is a sodium salt. In some embodiments, contact of the formulation with a biological sample results in one or more of the following: (a) inactivation of a nuclease in the biological sample, (b) inactivation of a pathogenic microbe in the biological sample, (c) disruption of an association of a protein with a nucleic acid in the biological sample, and (d) reduction or prevention of degradation of a nucleic acid in the biological sample, wherein the biological sample is derived from a subject.

In some embodiments, the formulations comprise a sodium salt of a detergent, a metal chelating agent and a buffer. In some embodiments, the concentration of the detergent is in the range of about 0.1% to about 5%, for example about 0.5%, about 1%, about 1.5%, about 2%, about 2.5%, about 3%, about 3.5%, about 4%, about 4.5%, or about 5%, including all values and subranges that lie therebetween. In some embodiments, the concentration of the detergent is in the range of about 1% to about 3%. In some embodiments, the concentration of the detergent is about 2%. In some embodiments, the concentration of the detergent is about 3%. The detergent may be a sodium salt of any detergent known in the art or discovered in the future, such as, for example, sodium salts of carboxylic acids (i.e., soaps), sodium salts of sulfonic acids, and sodium salts of sulfuric acid. In some embodiments, the detergent is selected from the group consisting of sodium lauryl sulfate, sodium taurodeoxycholate, sodium taurocholate, sodium glycocholate, sodium deoxycholate, sodium cholate, and sodium alkylbenzene sulfonate. In some embodiments, the detergent is sodium lauryl sulfate.

In some embodiments, the concentration of the metal chelating agent is in the range of about 0.1 mM to about 5 mM, for example, about 0.5 mM, about 1 mM, about 1.5 mM, about 2 mM, about 2.5 mM, about 3 mM, about 3.5 mM, about 4 mM, about 4.5 mM, or about 5 mM, including all values and subranges that lie therebetween. In some embodiments, the concentration of the metal chelating agent is in the range of about 0.5 mM to about 1.5 mM. In some embodiments, the concentration of the metal chelating agent is about 1 mM. In some embodiments, the metal chelating agent is selected from the group consisting of ethylene glycol tetraacetic acid (EGTA), hydroxyl ethylethylenediaminetriacetic acid, di ethyl ene tri amine penta acetic acid, N,N-bis(carboxymethyl)glycine, ethylene diamine tetraacetic acid (EDTA), citrate anhydrous, sodium citrate, calcium citrate, ammonium citrate, ammonium bicitrate, citric acid, diammonium citrate, ferric ammonium citrate, and lithium citrate. In some embodiments, the metal chelating agent is EDTA, EGTA, or a combination thereof. In some embodiments, the formulation comprises 1 mM EDTA. In some embodiments, the formulation comprises 1 mM EGTA. In some embodiments, the formulation comprises 1 mM of EDTA and 1 mM of EGTA.

In some embodiments, the formulation comprises a buffer selected from the group consisting of tris(hydroxymethyl) aminomethane (Tris), citrate, 2-(N-morpholino)ethanesulfonic acid (MES), N,N-Bis(2-hydroxy-ethyl)-2-aminoethanesulfonic Acid (BES), 1,3-bis(tris(hydroxymethyl)methylamino)propane (Bis-Tris), 4-(2-hydroxyethyl)-1-piperazineethanesulfonic acid (HEPES), 3-(N-morpholino)propane sulfonic acid (MOPS), N,N-bis(2-hydroxyethyl)glycine (Bicine), N-[tris(hydroxymethyl)methyl]glycine (Tricine), N-2-acetamido-2-iminodiacetic acid (ADA), N-(2-Acetamido)-2-aminoethanesulfonic acid (ACES), piperazine-1,4-bis(2-ethanesulfonic acid) (PIPES), bicarbonate, and phosphate. In some embodiments, the buffer comprises tris(hydroxymethyl)aminomethane, citrate, 2-(N-morpholino)ethanesulfonic acid, N,N-Bis(2-hydroxyethyl)-2-aminoethanesulfonic acid, 1,3-bis(tris(hydroxymethyl)methyl amino)propane, 4-(2-hydroxyethyl)-1-piperazine ethane sulfonic acid, 3-(N-morpholino) propanesulfonic acid, bicarbonate, or phosphate. In some embodiments, the buffer comprises a phosphate buffer. In some embodiments, the concentration of the buffer is in the range of about 5 mM to about 60 mM, for example, about 10 mM, about 15 mM, about 20 mM, about 25 mM, about 30 mM, about 35 mM, about 40 mM, about 45 mM, about 50 mM, about 55 mM, about 60 mM, including all values and subranges that lie therebetween. In some embodiments, the concentration of the buffer is in the range of about 20 mM to about 40 mM. In some embodiments, the concentration of the buffer is about 30 mM.

In some embodiments, the formulation has a pH in the range of about 5.5 to about 8.5, for example about 6, about 6.1, about 6.2, about 6.3, about 6.4, about 6.5, about 6.6, about 6.7, about 6.8, about 6.9, about 7, about 7.5, about 8, or about 8.5 inclusive of all values and subranges that lie therebetween. In some embodiments, the formulation has a pH in the range of about 6.5 to about 7.5. In some embodiments, the formulation has a pH of about 6.7.

In some embodiments, the formulations disclosed herein comprise one or more short-chain alkanols, such as, for example, linear or branched-chain alcohols, such as, methanol, ethanol, propanol, butanol, pentanol, hexanol, or any combination thereof. In some embodiments, the short-chain alkanols are present in the formulation in an amount from about 1% to about 50%, for example about 5%, about 10%, about 15%, about 20%, about 25%, about 30%, about 35%, about 40%, about 45%, about 50%, including all values and subranges that lie therebetween. In some embodiments, the formulations disclosed herein comprise betaine, bovine serum albumin, or osmolytes such as trehalose, sorbitol, and the like. In some embodiments, the formulations further comprise one or more anti-microbial, anti-viral or anti-fungal agents known in the art.

In some embodiments, the formulation does not comprise a chaotropic agent or chaotrope. In some embodiments, the formulation comprises a chaotropic agent or chaotrope. Exemplary chaotropes include, without limitation, guanidine thiocyanate (GuSCN), guanidine hydrochloride (GuHCl), guanidine isothionate, potassium thiocyanate (KSCN), sodium iodide, sodium perchlorate, urea, or any combination thereof. Additional exemplary chaotropes and chaotropic salts are described in U.S. Pat. No. 5,234,809, which is incorporated herein by reference in its entirety for all purposes. In some embodiments, the chaotropic agent is selected from the group consisting of n-butanol, ethanol, guanidine isocyanate, guanidine hydrochloride, guanidinium thiocyanate, guanidinium chloride, lithium perchlorate, lithium acetate, magnesium chloride, phenol, 2-propanol, thiourea, and urea. In some embodiments, the chaotropic agent is guanidinium thiocyanate. In some embodiments, the formulation does not comprise a reducing agent. In some embodiments, the formulation comprises a reducing agent. In some embodiments, the reducing agent is selected from the group consisting of 2-mercaptoethanol, tris(2-carboxyethyl)phosphine, formamide, dithiothreitol, dimethylsulfoxide, and tris(2-carboxyethyl)phosphine.

In some embodiments, bringing the formulation in contact with bleach does not produce a hazardous compound. In some embodiments, the hazardous compound is hydrogen cyanide. In some embodiments, the formulations disclosed herein contain a defoaming agent. As used herein, a defoaming agent is capable of preventing the formation of bubbles that typically result from the presence of detergents in the formulation. Defoaming agents facilitate pipetting and handling of the formulations disclosed herein. Exemplary surfactants/defoaming agents include, without limitation, cocoamidopropyl hydroxysultaine, alkylaminopropionic acids, imidazoline carboxylates, betaines, sulfobetaines, sultaines, alkylphenol ethoxylates, alcohol ethoxylates, polyoxyethylenated polyoxypropylene glycols, polyoxyethylenated mercaptans, long-chain carboxylic acid esters, alkonolamides, tertiary acetylenic glycols, polyoxyethylenated silicones, N-alky-lpyrrolidones, alkylpolyglycosidases, silicone polymers such as Antifoam A®, or polysorbates such as Tween®, or any combination thereof.

In some embodiments, the biological sample comprises a nasal sample, an oral sample, a saliva sample, a urine sample, a stool sample, a bronchoalveolar lavage sample, a nasopharyngeal aspirate sample or a blood sample. In some embodiments, the biological sample comprises a nasal sample, an oral sample, a bronchoalveolar lavage sample, a nasopharyngeal aspirate sample or a saliva sample. In some embodiments, the biological sample is obtained from a nasopharyngeal swab, an oropharyngeal swab, or a combination thereof.

In some embodiments, the biological sample comprises, or is suspected to comprise a nucleic acid derived from a pathogenic microbe. In some embodiments, the biological sample comprises, or is suspected to comprise a pathogenic microbe. In some embodiments, the subject has or is at a risk of developing a disease associated with, correlated with, or caused by a pathogenic microbe. In some embodiments, contact of the formulation with the biological sample results in inactivation of a pathogenic microbe in the biological sample. In some embodiments, the pathogenic microbe is capable of causing disease in a healthy subject. In some embodiments, the inactivated pathogenic microbe is incapable of causing disease in the healthy subject. In some embodiments, the pathogenic microbe is a bacterium, fungus, virus, or parasite. In some embodiments, the pathogenic microbe is a virus. In some embodiments, the virus is selected from the group consisting of SARS-CoV-2, SARS-CoV-1, MERS-CoV, chikungunya virus, African Swine Fever virus, Dengue virus, Zika virus, Influenza virus A, Influenza virus B, Influenza virus C, Human Immunodeficiency Virus (HIV), Ebola virus, Hepatitis virus A, Hepatitis virus B, Hepatitis virus C, Hepatitis virus D, and Hepatitis virus E, herpes simplex virus type 1 (HSV-1), herpes simplex virus type 2 (HSV-2) and Human Papillomavirus. In some embodiments, the virus is SARS-CoV2.

In some embodiments, contact of the formulation with the biological sample results in inactivation of a nuclease in the biological sample. In some embodiments, the nuclease is an endogenous nuclease (that is, present in the biological sample), or an exogenous nuclease (that is, present in a source other than the biological sample). In some embodiments, the nuclease is an RNase or a DNase. In some embodiments, contact of the formulation with the biological sample results in disruption of an association of a protein with a nucleic acid in the biological sample. In some embodiments, the association of the protein with the nucleic acid comprises a physical binding of the protein with the nucleic acid. Thus, in some embodiments, the contact of the formulation with the biological sample results in isolation of the naked or free nucleic acids, which are not bound to proteins, lipids, polysaccharides, cellular components and debris, or other molecules originally present in the biological sample. The formulations disclosed herein are also capable of stabilizing the naked or free nucleic acids released from the sample by preventing hydrolysis or nuclease degradation.

In some embodiments, contact of the formulation with a biological sample results in the reduction or prevention of degradation of a nucleic acid in the biological sample. In some embodiments, contact of the formulation with a biological sample results in the reduction or prevention of degradation of naked or free nucleic acids in the biological sample. In some embodiments, the contact of the formulation with the biological sample reduces or prevents the degradation of at least about 50% (for example about 55%, about 60%, about 65%, about 70%, about 75%, about 80%, about 85%, about 90%, about 95%, about 86%, about 97%, about 98%, about 99%, or about 100%, including all subranges and values that lie therebetween) of the nucleic acids in the biological sample. In some embodiments, degradation of the nucleic acid is reduced or prevented for at least about 1 day, for example, about 1 day, about 2 days, about 3 days, about 4 days, about 5 days, about 6 days, about 10 days, about 1 week, about 2 weeks, about 3 weeks, about 1 month, about 2 months, about 3 months, about 4 months, about 5 months, about 6 months, about 1 year, or about 2 years, including all values and subranges that lie therebetween. In some embodiments, degradation of the nucleic acid is reduced or prevented during collection, lysis, storage, transport, and/or downstream processing of the biological sample.

In some embodiments, degradation of the nucleic acid is reduced or prevented for at least about 1 day at any temperature, for example at about −20° C., about −15° C., about −10° C., about −5° C., about 0° C., about 4° C., about 5° C., about 10° C., about 15° C., about 20° C., about 25° C., about 30° C., about 35° C., or about 40° C., including all values and subranges that lie therebetween. In some embodiments, degradation of the nucleic acid is reduced or prevented for at least about 1 day at room temperature. As defined herein, room temperature is a temperature in the range of about 18° C. to 26° C., for example, about 19° C., about 20° C., about 21° C., about 22° C., about 23° C., about 24° C., about 25° C., including all values and subranges that lie therebetween. In some embodiments, degradation of the nucleic acid is reduced or prevented over extreme temperature variations during storage and/or transport. In some embodiments, degradation of the nucleic acid is reduced or prevented for at least about 3 days at room temperature. In some embodiments, degradation of the nucleic acid is reduced or prevented for at least about 1 month at room temperature.

In some embodiments, the nucleic acid is derived from a pathogenic microbe. In some embodiments, the nucleic acid is an RNA, a DNA, or a combination thereof. In some embodiments, the nucleic acid is an RNA. In some embodiments, the RNA is derived from SARS-CoV-2 genomic RNA. In some embodiments, the RNA is SARS-CoV-2 genomic RNA.

In certain embodiments, the formulation comprises supplemental nucleic acids (e.g. RNA and/or DNA) which are beneficial for one or more the methods disclosed herein. First, the supplemental nucleic acids may aid in the isolation and the purification of the nucleic acid in the biological sample by acting as a “carrier” and increasing the yield of the purified nucleic acids. Second, supplemental nucleic acids may act as a positive control for downstream methods and to monitor the fidelity of nucleic acid isolation and purification. Third, the supplemental nucleic acids may act as a comparator or a calibrator for downstream quantitative analysis using techniques such as qRT-PCT, or any other technique known in the art for the purpose. The amount of supplemental nucleic acids added to the formulation may be in the range of about 1 μg to about 1 μg.

The disclosure provides formulations, comprising: (a) about 3% sodium lauryl sulfate; (b) about 1 mM each of the EDTA and EGTA; and (c) about 30 mM of phosphate buffer; wherein the formulation has a pH of about 6.7, and wherein the formulation does not comprise guanidinium thiocyanate. The formulations disclosed herein may be a liquid, such as a solution, a suspension (e.g., colloidal suspension), slurry, emulsion, homogenate, or the like.

The disclosure further provides containers comprising at least one of the formulations disclosed herein. The disclosure also provides sample collection systems, comprising: a collection device or a collection vessel; and at least one of the formulations disclosed herein. In some embodiments, the collection device or the collection vessel contains the formulation. The disclosure further provides kits comprising at least one of the formulations disclosed herein, and/or any one of the containers disclosed herein, and/or any one of the sample collection systems disclosed herein.

A sample collection device is any device or product that is used to obtain or isolate the biological sample from its surroundings, such as the subject or the environment. In some embodiments, the sample collection device comprises a swab, a curette, or culture loop; and a collection vessel, such as a vial test tube, or specimen cup, that contains one or more of the formulations disclosed herein. In some embodiments, the collection vessel is opened to insert the formulations disclosed herein, opened to insert the sample and optionally a portion of the collection device and closed for storage and/or transport. The collection vessel may be closed using a screw cap, snap top, press-and-turn top, or the like. In some embodiments, the kits disclosed herein further comprise one or more additional reagents, storage devices, transport devices, and/or instructions for obtaining, collecting, lysing, storing, or transporting samples. In some embodiments, the sample collection device is a tube containing the formulation, a swab, and/or a saliva collection device. In some embodiments, the saliva collection device is compatible with the transport tube. In some embodiments, the swab is not degraded when in contact with the transport media for a period of time. The period of time may be up to about 1 day, up to about 5 days, up to about 1 week, up to about 2 weeks, up to about 3 weeks, up to about 1 month, up to about 2 months, up to about 3 months, or up to about 6 months. In some embodiments, the swab is not degraded when in contact with the transport media for a period of time at room temperature.

In some embodiments, the kits disclosed herein further comprise one or more extraction devices to help isolate and/or purify the nucleic acids from the biological sample for downstream analyses. In some embodiments, the kits disclosed herein comprise a container such as a vial, test tube, flask, bottle, specimen cup, or other container, into which one or more of the formulations disclosed herein may be placed, and/or suitably aliquoted for individual specimen collection, transport, and storage. In some embodiments, the kits comprise a larger container, such as a case, that comprises the containers disclosed herein, and/or the collections devices disclosed herein, along with other equipment, instructions, and the like. In some embodiments, the kits comprise one or more additional reagents, buffers, or compounds, and may also further optionally include instructions for use of the kit in the collection of a clinical, diagnostic, environmental, or forensic sample, as well as instructions for the storage and transport of the biological sample in contact with the formulations of the disclosure. In some embodiments, the kits may comprise more than one collection device and collection vessel and any other components to be included, so that the kits can be used to collect multiple samples from the same source or different sources.

### Methods of Preparing and Using the Formulations

The disclosure further provides methods of preparing a composition, comprising (a) collecting a biological sample from a subject, and (b) bringing the biological sample in contact with any one of the formulations disclosed herein to form the composition.

The disclosure provides methods of transporting a biological sample derived from a subject, comprising (a) collecting the biological sample from the subject; (b) bringing the biological sample in contact with any one of the formulations disclosed herein to form a composition; and (c) transporting the composition. In some embodiments, step (b) results in one or more of the following: (a) inactivation of a nuclease in the biological sample, (b) inactivation of a pathogenic microbe in the biological sample, (c) disruption of an association of a protein with a nucleic acid in the biological sample, and (d) reduction or prevention of degradation of a nucleic acid in the biological sample. In some embodiments, steps (a) through (d) occur sequentially in any order. In some embodiments, steps (a) through (d) occur concurrently or simultaneously.

The disclosure provides methods of inactivating a pathogenic microbe in a biological sample derived from a subject, comprising bringing the biological sample in contact with any one of the formulations disclosed herein to form a composition. In some embodiments, the pathogenic microbe is capable of causing disease in a healthy subject. In some embodiments, the inactivated pathogenic microbe is incapable of causing disease in the healthy subject. In some embodiments, the pathogenic microbe is a bacterium, fungus, virus, or parasite. In some embodiments, the pathogenic microbe is a virus. In some embodiments, the virus is selected from the group consisting of SARS-CoV-2, SARS-CoV-1, MERS-CoV, chikungunya virus, African Swine Fever virus, Dengue virus, Zika virus, Influenza virus A, Influenza virus B, Influenza virus C, Human Immunodeficiency Virus (HIV), Ebola virus, Hepatitis virus A, Hepatitis virus B, Hepatitis virus C, Hepatitis virus D, and Hepatitis virus E, herpes simplex virus type 1 (HSV-1), herpes simplex virus type 2 (HSV-2) and Human Papillomavirus. In some embodiments, the virus is SARS-CoV2.

The disclosure provides methods of using the formulations disclosed herein for point-of-care testing, field studies, in-home health care or testing, triage/emergency and casualty assessment(s), mobile forensics, pathology, epidemiological sampling, crime scene investigation, paternity testing, pre- and post-pregnancy genetic screening, rape and incest testing and family counseling, confidential screening and testing for sexually transmitted diseases, including, without limitation, HIV, syphilis, chlamydia, gonorrhoeae, or other venereal diseases and the like, and may be used during the monitoring, etiology, and control of epidemic or pandemic diseases in both human and animal populations. The disclosure provides uses for the formulations disclosed herein in collecting and analyzing microbial (e.g., viral) samples to predict, manage shift and drift, and thus, manage an imminent or ongoing pandemic, such as the COVID-19 global pandemic.

The disclosure provides methods of detecting a nucleic acid in a biological sample, comprising (a) collecting the biological sample from a subject, (b) bringing the biological sample in contact with any one of the formulations disclosed herein to form a composition, and (c) detecting the nucleic acid in the composition. The disclosure further provides methods of purifying a nucleic acid from a biological sample, comprising (a) collecting the biological sample from a subject, (b) bringing the biological sample in contact with any one of the formulations disclosed herein to form a composition, and (c) purifying the nucleic acid from the composition.

In some embodiments, any one of the methods disclosed herein comprises purifying the nucleic acid. Purifying the nucleic acid may be done using any method that is known in the art for the purpose. For instance, purifying the nucleic acid may be done using any method that is disclosed in Ali et al. BioMed Research International, 2017, the contents of which are incorporated herein by reference in its entirety for all purposes. In some embodiments, purifying the nucleic acid comprises Boom extraction. In some embodiments, Boom extraction comprises bringing the nucleic acid in contact with silica. In some embodiments, purifying the nucleic acid comprises hybridization of the nucleic acid. In some embodiments, the nucleic acid is derived from a pathogenic microbe. In some embodiments, the nucleic acid is an RNA, a DNA, or a combination thereof. In some embodiments, the nucleic acid is an RNA. In some embodiments, the RNA is a SARS-CoV-2 genomic RNA.

In some embodiments, the nucleic acids isolated by the methods of disclosed herein are subjected to downstream processing steps, such as amplification or any process that involves the nucleic acids serve as templates. In certain embodiments, the nucleic acids isolated by the methods of disclosed herein serve as a template in one or more subsequent molecular biological applications, assays, or techniques, such as, for example, genetic fingerprinting; amplified fragment length polymorphism (AFLP) polymerase chain reaction (PCR); restriction fragment length polymorphism analysis (RFLP); allele-specific oligonucleotide analysis (ASOA); microsatellite analysis; Southern hybridization; Northern hybridization; variable number of tandem repeats (VNTR) PCR; dot-blot hybridization; quantitative real-time PCR; polymerase cycling assembly (PCA); nested PCR; quantitative PCR (Q-PCR); asymmetric PCR; DNA footprinting; single nucleotide polymorphism (SNP) genotyping; reverse transcription PCR (RT-PCR); multiplex PCR (m-PCR); multiplex ligation-dependent probe amplification (MLPA); ligation-mediated PCR (LmPCR); methylation specific PCR (MPCR); helicase-dependent amplification (HDA); overlap-extension PCR (OE-PCR); whole-genome amplification (WGA); plasmid isolation; allelic amplification; site-directed mutagenesis; high-throughput genetic screening; reverse transcription PCR (RT-qPCR) or any combination thereof.

In some embodiments, any one of the methods disclosed herein comprises transporting and/or storing the composition at room temperature. In some embodiments, any one of the methods disclosed herein comprises transporting and/or storing the composition at room temperature for at least 1 day. In some embodiments, any one of the methods disclosed herein comprises transporting and/or storing the composition at room temperature for at least 1 month. In some embodiments, any one of the methods disclosed herein comprises amplifying the nucleic acid. In some embodiments, any one of the methods disclosed herein comprises amplifying the nucleic acid after storing the composition at room temperature for at least 1 day. In some embodiments, any one of the methods disclosed herein comprises amplifying the nucleic acid after storing the composition at room temperature for at least 3 days. In some embodiments, any one of the methods disclosed herein comprises amplifying the nucleic acid after storing the composition at room temperature for at least 1 month.

All papers, publications and patents cited in this specification are herein incorporated by reference as if each individual paper, publication or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

### EXAMPLES

The following examples, which are included herein for illustration purposes only, are not intended to be limiting.

### Example 1: SARS-CoV-2 RNA Targets can be Detected in Biological Samples Preserved in Formulation D

Pathogen transport media formulations disclosed herein were developed based upon the chemical property of detergent SDS and its effects on biological specimen including lysis and nuclease inhibition. Four pathogen transport media formulations (Formulation A, B, C and D) with SDS concentration of 0.5%, 1%, 2% and 3%, respectively, were prepared in sodium phosphate buffer. Each of four transport tubes were filled with 1.5 mL of the Formulation A, B C, or D. In this study, it was evaluated whether the presence of SARS-CoV-2 RNA in a biological sample stored in the pathogen transport media formulations disclosed herein can be detected using standard methodology. SARS-CoV-2 RNA (Twist Biosciences) was purchased at stock concentration of 1 million copies/μL. The stock solution was diluted by adding 2 μL of the stock solution to 198 μL nuclease acid free water to arrive at a concentration of 10,000 copies/μL. 10 μL aliquots were prepared and stored at −80° C. for all development test process. Any remaining aliquot was discarded after two freeze-thaw cycles.

15 μL pooled healthy donor serum was added as internal human specimen control (HSC) to each of the transport tubes containing 1.5 mL Formulation A, B C, or D. The mixture was mixed by pulse vortex and spun. The tubes were stored at room temperature for 10 minutes. 1.5 μL of 10,000 copies/μL SAR-CoV-2 RNA solution was added to each of the transport tubes containing Formulation A, B C, or D and HSC to achieve 10 copies/O_, SAR-CoV-2 RNA. 1.5 μL nuclease free water was used to replace RNA as negative controls. The tubes were mixed by pulse vortex and spun. The tubes were stored at room temperature for 1 hour, 24 hours and 72 hours, followed by nucleic acid extraction.

At the extraction time, 2000_, of each of the above-described samples was transferred to a clean deep 96 well plate for RNA extraction following the Thermo Fisher MagMax viral/pathogen nucleic acid isolation kit protocol. 5 μL of final extraction eluate was used for RT-qPCR following the CDC 2019-nCoV TaqMan assay EUA protocol with TaqPath one-step RT-qPCR mix (Thermo Fisher Scientific). The CDC 2019-nCoV TaqMan assay contains the three primer/probe mixes, including two mixes that detect fragments of nucleoprotein (N) gene (N1 and N2) in the SAR-COV-2 genomic RNA, as well as, one primer/probe mix that detects human RNase P RNA as an internal process control. The RT-qPCR runs were carried out on the Thermo Fisher Quant Studio 5 (e.g. QS 5) instrument and data were acquired with instrument analysis software with default setting parameters. All tested contrived samples showed valid positive detection (that is, cycle threshold (CT) less than 40), while the negative controls were valid negatives (Table 1).

TABLE 1
CT values of samples described above indicate successful SARS-CoV-2 RNA detection
in Formulations A (0.5% SDS), B (1% SDS), C (2% SDS), and D (3% SDS).

| | Sample | Target | | | |
|---|---|---|---|---|---|
| | Name | Name | CT/1 Hr | CT/24 Hr | CT/72 Hr |
| SDS Formula + | 0.5% | N1 | 34.1 | 34.4 | 34.3 |
| K1:P31 | SDS | N1 | 34.6 | 34.0 | 34.2 |
| | | N2 | 34.5 | 35.0 | 34.9 |
| | | N2 | 35.6 | 35.2 | 34.4 |
| | | RNaseP | 34.3 | 34.3 | 33.8 |
| | | RNaseP | 34.4 | 35.1 | 33.6 |
| | 1% SDS | N1 | 35.8 | 35.7 | 34.6 |
| | | N1 | 34.3 | 34.8 | 34.4 |
| | | N2 | 35.4 | 35.1 | 34.9 |
| | | N2 | 35.0 | 36.1 | 34.6 |
| | | RNaseP | 34.5 | 33.7 | 34.0 |
| | | RNaseP | 34.3 | 33.9 | 33.2 |
| | 2% SDS | N1 | 34.3 | 34.2 | 34.2 |
| | | N1 | 34.8 | 34.7 | 34.3 |
| | | N2 | 34.3 | 35.4 | 34.7 |
| | | N2 | 35.4 | 34.6 | 34.9 |
| | | RNaseP | 33.5 | 33.6 | 33.7 |
| | | RNaseP | 33.8 | 33.8 | 33.8 |
| | 3% SDS | N1 | 34.3 | 34.2 | 35.0 |
| | | N1 | 34.7 | 34.0 | 35.4 |
| | | N2 | 35.3 | 35.4 | 35.3 |
| | | N2 | 35.5 | 35.8 | 34.3 |
| | | RNaseP | 34.4 | 33.1 | 34.3 |
| | | RNaseP | 34.6 | 34.3 | 33.6 |
| | 3% SDS | N1 | Undetermined | Undetermined | Undetermined |
| | serum | N1 | Undetermined | Undetermined | Undetermined |
| | only no | N2 | Undetermined | Undetermined | Undetermined |
| | Covid | N2 | Undetermined | Undetermined | Undetermined |
| | RNA | RNaseP | 33.832 | 33.2 | 32.4 |
| | | RNaseP | 34.454 | 33.1 | 33.2 |
| Assay | Water | N1 | Undetermined | Undetermined | Undetermined |
| Controls | | N1 | Undetermined | Undetermined | Undetermined |
| | | N2 | Undetermined | Undetermined | Undetermined |
| | | N2 | Undetermined | Undetermined | Undetermined |
| | | RNaseP | 38.3** | Undetermined | Undetermined |
| | | RNaseP | 36.8** | Undetermined | Undetermined |
| | RT-qPCR | N1 | Undetermined | Undetermined | |
| | NTC | N1 | Undetermined | Undetermined | |
| | | N2 | Undetermined | Undetermined | |
| | | N2 | Undetermined | Undetermined | |
| | | RNaseP | Undetermined | Undetermined | |
| | | RNaseP | Undetermined | Undetermined | |
| | Pos20 or | N1 | 37.3 | 33.8 | |
| | 200 cps | N1 | 37.7 | 34.7 | |
| | | *N2 | Undetermined | 36.6 | |
| | | *N2 | Undetermined | 34.6 | |
| | | RNaseP | Undetermined | Undetermined | |
| | | RNaseP | Undetermined | Undetermined | |

*NTC = No template control;
POS = positive control

Furthermore, Table.1 shows that there is no significant degradation of RNA target over 72 hours even at room temperature when the formulations disclosed herein are used. Also, there is no significant difference in the amount of target detected, as indicated by the CT values, with SDS concentration. These data indicate that all four pathogen transport media formulations (Formulation A, B, C and D) stabilized viral RNA target for successful detection up to 72 hours. Formulations C and D with the higher SDS concentration (2% and 3%) were selected for a detection sensitivity study.

### Example 2: Detection Sensitivity of Biological Samples Preserved in Formulation D

In this study, the sensitivity of detection of SARS CoV-2 RNA which has been stored in the pathogen transport media formulations disclosed herein was evaluated in the following manner. 15 μL of pooled healthy donor serum was added as internal human specimen control (HSC) to each of the transport tubes containing 1.5 mL of Formulation C or Formulation D. The contents of the tubes were mixed by pulse vortex and spun, and stored at room temperature for 10 minutes. 1.5 μL of 10,000 copies/μL SARS-CoV-2 RNA was diluted in 8.5 μL or 98.5 μL nuclease free water to achieve 1500 copies/μL or 150 copies/μL of SARS-CoV-2 RNA. 5 μL, 2 μL, or 1μL of 1500 copies/μL SAR-CoV-2 RNA solution was added to each of the transport tubes containing 1.5 mL of Formulation C or Formulation D and 15 μL HSC to achieve 5 copies/μL, 2 copies/μL, and 1 copy/μL SARS-CoV-2 RNA, respectively. Additionally, 5 μL or 0.5 μL of 100 copies/μL SARS-CoV-2 RNA solution was added to each of the transport tubes containing 1.5 mL of Formulation C or Formulation D and 15 μL HSC to achieve 0.5 copies/μL, and 0.05 copies/μL SARS-CoV-2 RNA, respectively. 1.5 μL nuclease free water was used to replace RNA as a negative controls. The tubes were mixed by pulse vortexing, spun, and stored at room temperature for 1 hour, 24 hours and 72 hours, followed by nucleic acid extraction. RNA extraction and RT-qPCR were performed as described in Example 1.

The results, tabulated in Table 2, showed surprisedly that even after 72 hours of storage at room temperature, all contrived samples with 5 copies/μL gave valid positive results, while samples with 1 or 2 copies/μL target RNA showed inconclusive results, with one of the two targets showing negative results. Based on these results, the detection sensitivity of SARS-CoV-2 RNA using MagMax Pathogen/viral nucleic acid extraction method was estimated to be between 1 to 5 copies/μL using this assay.

TABLE 2
CT values for detection and sensitivity testing of samples

| SDS formula | | | | | | | |
|---|---|---|---|---|---|---|---|
| RNA spike | Taqman | Formulation C | | | Formulation D | | |
| cp/ul | Target | CT/2 Hrs | CT/Day 1 | CT/Day 3 | CT/2 Hrs | CT/Day 1 | CT/Day 3 |
| 5.00 | N1 | 36.5 | 36.7 | 36.1 | 35.1 | 36.0 | 35.6 |
| | N1 | 35.8 | 37.0 | 37.4 | 35.1 | 34.6 | 35.7 |
| | N2 | 36.3 | 36.2 | 40.5 | 36.4 | 34.0 | 34.9 |
| | N2 | 36.0 | 36.0 | 41.4 | 35.2 | 34.7 | 35.6 |
| | RNaseP | 34.0 | 33.5 | 33.3 | 33.5 | 33.7 | 33.9 |
| | RNaseP | 33.8 | 33.5 | 33.9 | 33.1 | 33.5 | 33.9 |
| 2.00 | N1 | 38.1 | UnD | 35.6 | UnD | 36.0 | 37.9 |
| | N1 | UnD | 37.9 | 36.6 | 36.3 | 36.3 | 36.9 |
| | N2 | 37.0 | 38.0 | 36.9 | 36.7 | 37.0 | 37.8 |
| | N2 | 36.9 | UnD | 36.3 | 36.9 | 36.1 | 37.2 |
| | RNaseP | 33.4 | 33.0 | 33.4 | 33.8 | 33.8 | 33.9 |
| | RNaseP | 34.0 | 33.4 | 33.2 | 33.8 | 33.7 | 33.6 |
| 1.00 | N1 | 38.0 | UnD | UnD | UnD | 37.3 | 37.0 |
| | N1 | 36.0 | 37.9 | 37.3 | UnD | 37.2 | UnD |
| | N2 | 39.4 | 38.6 | 35.6 | 39.1 | 36.7 | 39.9 |
| | N2 | 38.3 | 39.2 | 37.5 | UnD | 36.6 | 38.1 |
| | RNaseP | 33.5 | 33.2 | 33.6 | 33.7 | 33.5 | 33.1 |
| | RNaseP | 34.1 | 33.5 | 34.0 | 33.6 | 34.0 | 33.3 |
| 0.50 | N1 | UnD | 37.9 | 37.3 | UnD | 37.4 | UnD |
| | N1 | UnD | UnD | 38.0 | UnD | UnD | UnD |
| | N2 | 39.0 | UnD | UnD | UnD | UnD | UnD |
| | N2 | UnD | UnD | UnD | 39.3 | UnD | UnD |
| | RNaseP | 34.2 | 33.7 | 33.2 | 33.6 | 34.1 | 33.0 |
| | RNaseP | 34.1 | 33.3 | 33.5 | 35.1 | 34.0 | 33.4 |
| 0.05 | N1 | UnD | UnD | UnD | UnD | 36.9 | UnD |
| | N1 | UnD | UnD | UnD | UnD | UnD | UnD |
| | N2 | UnD | UnD | UnD | UnD | UnD | UnD |
| | N2 | UnD | UnD | UnD | UnD | UnD | UnD |
| | RNaseP | 33.3 | 33.5 | 33.5 | 34.1 | 33.5 | 33.3 |
| | RNaseP | 33.2 | 33.6 | 33.6 | 33.8 | 33.6 | 33.5 |
| HSC | N1 | UnD | UnD | UnD | UnD | UnD | UnD |
| serum only | N1 | UnD | UnD | UnD | UnD | UnD | UnD |
| | N2 | UnD | UnD | UnD | UnD | UnD | UnD |
| | N2 | UnD | UnD | UnD | UnD | UnD | UnD |
| | RNaseP | 34.0 | 34.1 | 34.0 | 33.4 | 33.8 | 33.6 |
| | RNaseP | 34.8 | 33.5 | 33.4 | 33.8 | 34.1 | 33.8 |

RT-qPCR Controls

| RNA spike | Taqman | | | | |
|---|---|---|---|---|---|
| cp/ul | Target | CT/2 Hrs | CT/Day 1 | CT/Day 3 | CT/Day 7 |
| NTC | N1 | UnD | UnD | UnD | UnD |
| | N1 | UnD | UnD | UnD | UnD |
| | N2 | UnD | UnD | UnD | UnD |
| | N2 | UnD | UnD | UnD | UnD |
| | RNaseP | UnD | UnD | UnD | UnD |
| | RNaseP | UnD | UnD | UnD | UnD |
| POS | N1 | 37.0 | 35.9 | UnD | 36.4 |
| (5 cps/ul) | N1 | 37.3 | 36.5 | 37.2 | 35.3 |
| | N2 | 36.0 | 37.2 | 36.4 | 37.7 |
| | N2 | 36.7 | 36.5 | 37.7 | 37.6 |
| | RNaseP | UnD | UnD | UnD | UnD |
| | RNaseP | UnD | UnD | UnD | UnD |

*NTC = No template control; POS = positive control

The CDC 2019-nCoV TaqMan assay utilized in the experiment above has a detection limit (LoD) of 1-3 copies/μL as validated by the CDC under the FDA's Emergency Usage Authorization. Based on these results, we estimate that the sensitivity of sample detection of samples preserved in Formulations C or D meets the designed assay detection sensitivity (See CDC 2019-nCoV TaqMan assay EUA analytical performance review for details). Further titration (e.g. 3 copies/μL) will be included in following performance evaluation studies.

### Example 3: Long Term Sample Storage in Various Formulations

In this study, the pathogen transport media formulations disclosed herein were further evaluated for their ability to preserve nucleic acids (such as, SARS-CoV-2 RNA) in a biological sample over a long period of time, such as several days or weeks.

15 μL pooled healthy donor serum was added as an internal human specimen control (HSC) to each of the transport tubes containing 1.5 mL of Formulation C and D. The tubes were mixed by pulse vortexing, spun, and stored at room temperature for 10 minutes. 1.5 μL of 10,000 copies/μL SARS-CoV-2 RNA was diluted in 8.5 μL or 98.5 μL nuclease free water to achieve 1500 copies/μL or 150 copies/μL 3 μL, 2 μL and 1 μL of 1500 copies/O_, SARS-CoV-2 RNA solution was added to each of the transport tubes containing 1.5 mL Formulation C or D, and 15 μL HSC added to achieve 3 copies/μL, 2 copies/μL and 1 copy/μL SARS-CoV-2 RNA, respectively. 5 μL or 0.5 μL of 100 copies/O_, SARS-CoV-2 RNA solution was added to each of the transport tubes containing Formulation C or D, and 15 μL HSC added to achieve 0.5 copies/μL, and 0.05 copies/μL, respectively. 1.5 μL nuclease free water was used to replace RNA as a negative control. The tubes were mixed by pulse vortexing, spun, and stored at room temperature for 2 hours, 1 day, 3 days, 7 days, 14 days, 21 days and 30 days prior to nucleic acid extraction. RNA extraction and RT-qPCR were performed as described in Example 1.

TABLE 3
Time course study of SARS-CoV-2 RNA storage in Formulation C or D at room temperature (CT values)

| SDS formula | | Formulation C | | | | | | | | |
|---|---|---|---|---|---|---|---|---|---|---|
| RNA spike | Taqman | | | | | | | | CT | CT |
| cp/ul | Target | CT/2 Hrs | CT/Day 1 | CT/Day 3 | CT/Day 7 | CT/Day 14 | CT/Day 21 | CT/Day 30 | GeoMean | Median |
| 5.00 | N1 | 36.5 | 36.7 | 36.1 | 36.5 | 35.2 | 36.3 | 34.5 | 36.0 | 36.3 |
| | N1 | 35.8 | 37.0 | 37.4 | 37.1 | 34.0 | 36.3 | 34.8 | | |
| | N2 | 36.3 | 36.2 | 40.5 | 36.7 | 34.8 | 34.9 | 34.9 | 36.5 | 36.1 |
| | N2 | 36.0 | 36.0 | 41.4 | 36.6 | 35.9 | 35.1 | 36.2 | | |
| | RNaseP | 34.0 | 33.5 | 33.3 | 34.4 | 33.4 | 32.6 | 33.9 | 33.6 | 33.6 |
| | RNaseP | 33.8 | 33.5 | 33.9 | 33.7 | 33.5 | 32.5 | 34.3 | | |
| 2.00 | N1 | 38.1 | UnD | 35.6 | 36.7 | 36.6 | 36.8 | 37.8 | 36.9 | 36.7 |
| | N1 | UnD | 37.9 | 36.6 | 37.0 | 37.3 | 36.2 | 36.3 | | |
| | N2 | 37.0 | 38.0 | 36.9 | 37.3 | 36.0 | 35.8 | UnD | 37.2 | 36.9 |
| | N2 | 36.9 | UnD | 36.3 | 40.0 | 39.2 | 36.6 | 36.8 | | |
| | RNaseP | 33.4 | 33.0 | 33.4 | 33.6 | 33.5 | 32.5 | 33.5 | 33.4 | 33.4 |
| | RNaseP | 34.0 | 33.4 | 33.2 | 34.0 | 34.3 | 32.5 | 33.8 | | |
| 1.00 | N1 | 38.0 | UnD | UnD | 37.8 | 36.9 | 37.3 | 35.8 | 37.3 | 37.5 |
| | N1 | 36.0 | 37.9 | 37.3 | UnD | 38.0 | UnD | 37.7 | | |
| | N2 | 39.4 | 38.6 | 35.6 | UnD | 38.5 | 35.8 | UnD | 38.3 | 38.3 |
| | N2 | 38.3 | 39.2 | 37.5 | UnD | 44.0 | 37.4 | 37.5 | | |
| | RNaseP | 33.5 | 33.2 | 33.6 | 33.8 | 33.1 | 31.8 | 34.0 | 33.4 | 33.6 |
| | RNaseP | 34.1 | 33.5 | 34.0 | 34.1 | 33.8 | 32.1 | 33.6 | | |
| 0.50 | N1 | UnD | 37.9 | 37.3 | UnD | UnD | UnD | 36.7 | 37.8 | 37.9 |
| | N1 | UnD | UnD | 38.0 | 37.9 | UnD | 38.9 | UnD | | |
| | N2 | 39.0 | UnD | UnD | 39.9 | UnD | UnD | UnD | 39.4 | 39.5 |
| | N2 | UnD | UnD | UnD | UnD | UnD | UnD | UnD | | |
| | RNaseP | 34.2 | 33.7 | 33.2 | 33.7 | UnD | 40.5 | 33.9 | 34.5 | 33.8 |
| | RNaseP | 34.1 | 33.3 | 33.5 | 33.4 | UnD | 37.2 | 33.8 | | |
| 0.05 | N1 | UnD | UnD | UnD | UnD | UnD | UnD | UnD | 36.8 | 36.8 |
| | N1 | UnD | UnD | UnD | UnD | UnD | 36.8 | UnD | | |
| | N2 | UnD | UnD | UnD | UnD | UnD | 40.6 | UnD | 39.6 | 39.6 |
| | N2 | UnD | UnD | UnD | UnD | UnD | 38.6 | UnD | | |
| | RNaseP | 33.3 | 33.5 | 33.5 | 33.4 | 33.6 | 32.9 | 34.3 | 33.5 | 33.5 |
| | RNaseP | 33.2 | 33.6 | 33.6 | 33.8 | 34.4 | 32.4 | 33.6 | | |
| HSC | N1 | UnD | UnD | UnD | UnD | UnD | UnD | UnD | #NUM! | #NUM! |
| serum only | N1 | UnD | UnD | UnD | UnD | UnD | UnD | UnD | | |
| | N2 | UnD | UnD | UnD | UnD | UnD | UnD | UnD | #NUM! | #NUM! |
| | N2 | UnD | UnD | UnD | UnD | UnD | UnD | UnD | | |
| | RNaseP | 34.0 | 34.1 | 34.0 | 33.5 | 33.3 | 35.0 | 33.6 | 34.0 | 33.9 |
| | RNaseP | 34.8 | 33.5 | 33.4 | 34.0 | 33.8 | 34.6 | 33.8 | | |
| SDS formula | | Formulation D | | | | | | | | |
| RNA spike | Taqman | | | | | | | | CT | |
| cp/ul | Target | CT/2 Hrs | CT/Day 1 | CT/Day 3 | CT/Day 7 | CT/Day 14 | CT/Day 21 | CT/Day 30 | GeoMean | Median |
| 5.00 | N1 | 35.1 | 36.0 | 35.6 | 35.5 | UnD_EE [symbol] | 34.6 | 34.9 | 35.3 | 35.6 |
| | N1 | 35.1 | 34.6 | 35.7 | 35.9 | UnD_EE | 35.2 | 35.6 | | |
| | N2 | 36.4 | 34.0 | 34.9 | 36.0 | UnD_EE | UnD | 35.7 | 35.5 | 35.6 |
| | N2 | 35.2 | 34.7 | 35.6 | 35.6 | UnD_EE | 35.0 | 36.8 | | |
| | RNaseP | 33.5 | 33.7 | 33.9 | 33.7 | UnD_EE | 31.5 | 34.0 | 33.3 | 33.7 |
| | RNaseP | 33.1 | 33.5 | 33.9 | 33.6 | UnD_EE | 32.0 | 33.7 | | |
| 2.00 | N1 | UnD | 36.0 | 37.9 | UnD | 37.1 | 35.2 | UnD | 36.8 | 36.3 |
| | N1 | 36.3 | 36.3 | 36.9 | UnD | 41. [symbol] | 36.4 | 34.9 | | |
| | N2 | 36.7 | 37.0 | 37.8 | 36.4 | 37.4 | 36.5 | 36.9 | 36.8 | 36.7 |
| | N2 | 36.9 | 36.1 | 37.2 | 36.3 | 37.5 | UnD | 36.0 | | |
| | RNaseP | 33.8 | 33.8 | 33.9 | 34.0 | 33.3 | 31.8 | 34.3 | 33.5 | 33.8 |
| | RNaseP | 33.8 | 33.7 | 33.6 | 33.6 | 33.6 | 32.1 | 33.8 | | |
| 1.00 | N1 | UnD | 37.3 | 37.0 | 36.3 | 37.1 | UnD | 37.0 | 36.8 | 37.1 |
| | N1 | UnD | 37.2 | UnD | 37.8 | UnD | 35.7 | 35.9 | | |
| | N2 | 39.1 | 36.7 | 39.9 | 37.3 | 37.6 | UnD | 37.4 | 38.1 | 38.1 |
| | N2 | UnD | 36.6 | 38.1 | 38.1 | 38.7 | 39.1 | 38.8 | | |
| | RNaseP | 33.7 | 33.5 | 33.1 | 34.2 | 33.5 | 32.4 | 34.3 | 33.5 | 33.7 |
| | RNaseP | 33.6 | 34.0 | 33.3 | 33.8 | 33.3 | 32.1 | 33.9 | | |
| 0.50 | N1 | UnD | 37.4 | UnD | UnD | UnD | 38.1 | UnD | 37.8 | 37.4 |
| | N1 | UnD | UnD | UnD | UnD | 37.9 | UnD | UnD | | |
| | N2 | UnD | UnD | UnD | 39.0 | 39.0 | UnD | UnD | 39.5 | 39.3 |
| | N2 | 39.3 | UnD | UnD | UnD | UnD | UnD | 40.5 | | |
| | RNaseP | 33.6 | 34.1 | 33.0 | 35.0 | 33.7 | 32.5 | 33.7 | 33.6 | 34.1 |
| | RNaseP | 35.1 | 34.0 | 33.4 | 34.1 | 33.2 | 32.0 | 34.1 | | |
| 0.05 | N1 | UnD | 36.9 | UnD | UnD | UnD | UnD | UnD | 36.9 | 36.9 |
| | N1 | UnD | UnD | UnD | UnD | UnD | UnD | UnD | | |
| | N2 | UnD | UnD | UnD | UnD | UnD | UnD | 38.7 | 38.7 | #NUM! |
| | N2 | UnD | UnD | UnD | UnD | UnD | UnD | UnD | | |
| | RNaseP | 34.1 | 33.5 | 33.3 | 33.3 | 34.0 | 31.9 | 34.3 | 33.5 | 33.6 |
| | RNaseP | 33.8 | 33.6 | 33.5 | 33.8 | 33.5 | 32.7 | 33.7 | | |
| HSC | N1 | UnD | UnD | UnD | UnD | UnD | UnD | UnD | #NUM! | #NUM! |
| serum only | N1 | UnD | UnD | UnD | UnD | UnD | UnD | UnD | | |
| | N2 | UnD | UnD | UnD | UnD | UnD | UnD | UnD | #NUM! | #NUM! |
| | N2 | UnD | UnD | UnD | UnD | UnD | UnD | UnD | | |
| | RNaseP | 33.4 | 33.8 | 33.6 | 34.4 | 33.9 | 32.0 | 34.0 | 33.5 | 33.8 |
| | RNaseP | 33.8 | 34.1 | 33.8 | 33.8 | 33.6 | 32.2 | 33.7 | | |
| RT-qPCR Controls | | | | | | | | | | |
| RNA spike | Tagman | | | | | | | | CT | |
| cp/ul | Target | CT/2 Hrs | CT/Day 1 | CT/Day 3 | CT/Day 7 | CT/Day 14 | CT/Day 21 | CT/Day 30 | GeoMean | Median |
| NTC | N1 | UnD | UnD | UnD | UnD | UnD | UnD | UnD | #NUM! | #NUM! |
| | N1 | UnD | UnD | UnD | UnD | UnD | UnD | UnD | | |
| | N2 | UnD | UnD | UnD | UnD | UnD | UnD | UnD | #NUM! | #NUM! |
| | N2 | UnD | UnD | UnD | UnD | UnD | UnD | UnD | | |
| | RNaseP | UnD | UnD | UnD | UnD | UnD | UnD | UnD | #NUM! | #NUM! |
| | RNaseP | UnD | UnD | UnD | UnD | UnD | UnD | UnD | | |
| POS | N1 | 37.0 | 35.9 | UnD | 36.4 | 37.2 | UnD_EE | 35.3 | 36.3 | 36.4 |
| (5 cps/ul) | N1 | 37.3 | 36.5 | 37.2 | 35.3 | 36.9 | UnD_EE | 34.4 | | |
| | N2 | 36.0 | 37.2 | 36.4 | 37.7 | 38.3 | UnD_EE | 35.6 | 36.9 | 36.7 |
| | N2 | 36.7 | 36.5 | 37.7 | 37.6 | 37.1 | UnD_EE | 36.1 | | |
| | RNaseP | UnD | UnD | UnD | UnD | UnD | UnD_EE | UnD | | |
| | RNaseP | UnD | UnD | UnD | UnD | UnD | UnD_EE | UnD | | |

EE [symbol] Experiment Error, invalid data
*NTC = No template control; POS = positive control [symbol]
indicates data missing or illegible when filed

As shown in Table 3 above, unexpectedly we found that SARS-CoV-2 RNA can be detected in samples preserved using Formulation C or D for up to a month. Additionally, our results show that mean CT values of samples stored in Formulation D are lower than those in Formulation C, indicating that the use of Formulation D potentially gives higher extraction yields and better downstream detection sensitivity.

### Example 4: Performance Comparison with a Commercial Kit (Zymo DNA/RNA Shield)

This study compares the performance of a commercially available kit (Zymo Research) with the performance of Formulation D disclosed herein. This example compares the performance of this commercially used kit with the performance of Formulation D disclosed here. The results show that Formulation D has far superior properties as compared to the commercially used alternative. 1 mL of Formulation D was used to fill the sample transport tube in order to match the volume of DNA/RNA Shield™ (Zymo Research). 10 μL pooled healthy donor serum was added as an internal human specimen control (HSC) to each of the transport tubes containing one of the following transport media: 1 mL Formulation D, 1 mL Formulation C or DNA/RNA Shield™ (Zymo Research). The tubes were mixed by pulse vortexing, spun, and stored at room temperature for 10 minutes.

2 μL of 10,000 copies/μL SARS-CoV-2 RNA was diluted in 18 μL or 1998 μL nuclease free water to achieve 1000 copies/μL or 10 copies/μL. 3 μL or 1 μL of 1000 copies/μL The SAR-CoV-2 RNA solution was added to each of the transport tubes containing 1.0 mL transport media and 10 μL HSC to achieve 3 copies/μL or 1 copy/μL SARS-CoV-2 RNA. 5 of 10 copies/μL SAR-CoV-2 RNA solution was added to each of the transport tubes containing 1.0 mL transport media and 10 μL HSC to achieve 0.05 copies/μL. 3 μL nuclease free water was added to replace RNA as negative controls. The tubes were mixed by pulse vortexing, spun, and stored at room temperature for 1 day, and 7 days, followed by nucleic acid extraction. RNA extraction and RT-qPCR analysis was performed as described in Example 1.

Additionally, performance comparisons were conducted with contrived clinical samples. Oropharyngeal (OP) swab samples were collected from two healthy volunteers following CDC recommended sample collection protocols. All samples were collected in tubes containing 1 mL of each of the transport media being tested. 2 μL 10,000 copies/μL SAR-CoV-2 RNA was diluted in 18 μL nuclease free water to achieve 1000 copies/μL RNA. 3 of 1000 copies/O_, SARS-CoV-2 RNA was added to each of above OP swab samples as contrived positive clinical samples. 3 μL nuclease free water was added to replace RNA as contrived negative clinical samples. All samples were stored at room temperature for 1 day and 7 days for follow on RNA extraction. RNA extraction and RT qPCR was performed as described in Example 1.

The results show that Formulation C and D had similar performance as DNA/RNA Shield™ sample collection kits (Zymo Research and Pangea Laboratory) with either contrived SARS-CoV-2 RNA samples or contrived clinical samples (oropharyngeal swab samples). See Tables 4 and 5.

TABLE 4
CT value comparison of Contrived RNA samples stored in Formulation D, Formulation C or DNA/RNA
Shield ™ sample collection kits (Zymo Research and Pangea Laboratory).

| Tube type | | Zymo | | | Formulation D | | | Formulation C | | |
|---|---|---|---|---|---|---|---|---|---|---|
| RNA input | Target | CT/24 hrs | CT/72 hrs | CT/Day 7 | CT/24 hrs | CT/72 hrs | CT/Day 7 | CT/24 hrs | CT/72 hrs | CT/Day 7 |
| 3 | N1 | 36.0 | 37.6 | UnD | 34.4 | 37.7 | 38.1 | 35.0 | 35.9 | 37.0 |
| cp/ul | N1 | 35.1 | UnD | UnD | 35.4 | 37.3 | 36.7 | 36.2 | 36.4 | 35.9 |
| | N2 | 39.3 | 35.4 | 37.7 | 36.8 | 38.6 | UnD | 36.8 | UnD | 37.0 |
| | N2 | 39.6 | 37.4 | UnD | 39.3 | 38.3 | 37.7 | 39.3 | 37.0 | 38.2 |
| | RNaseP | 36.1 | 35.4 | 34.9 | 34.1 | 34.6 | 34.4 | 34.0 | 34.0 | 33.5 |
| | RNaseP | 34.8 | 34.8 | UnD | 34.6 | 34.8 | 33.5 | 33.6 | 34.7 | 34.2 |
| 1 | N1 | 37.3 | 37.9 | UnD | UnD | UnD | 38.0 | 35.5 | 37.7 | UnD |
| cp/ul | N1 | UnD | UnD | NA | 37.0 | 36.0 | UnD | 36.1 | 36.2 | 38.1 |
| | N2 | UnD | 38.6 | 38.4 | UnD | UnD | 37.8 | UnD | 38.4 | 37.8 |
| | N2 | 41.8 | UnD | NA | 39.1 | UnD | 37.7 | 38.0 | 38.9 | UnD |
| | RNaseP | 34.8 | 35.6 | 35.6 | 34.6 | 34.1 | 33.6 | 34.3 | 34.0 | 34.7 |
| | RNaseP | 34.9 | 34.5 | NA | 34.6 | 33.9 | 34.3 | 34.0 | 34.0 | 34.1 |
| 0.05 | N1 | UnD | UnD | UnD | UnD | UnD | UnD | UnD | UnD | UnD |
| cp/ul | N1 | UnD | UnD | NA | UnD | UnD | UnD | UnD | UnD | UnD |
| | N2 | UnD | UnD | UnD | UnD | UnD | UnD | UnD | UnD | UnD |
| | N2 | UnD | UnD | 37.6 | UnD | UnD | UnD | UnD | UnD | UnD |
| | RNaseP | 36.0 | 35.1 | NA | 35.2 | 34.6 | 34.4 | 34.6 | 33.5 | 34.4 |
| | RNaseP | 35.4 | 34.1 | | 34.8 | 34.2 | 34.1 | 33.7 | 34.1 | 33.7 |
| 0 | N1 | UnD | UnD | UnD | UnD | UnD | UnD | UnD | UnD | UnD |
| cp/ul | N1 | UnD | UnD | NA | UnD | UnD | UnD | UnD | UnD | UnD |
| | N2 | UnD | UnD | UnD | UnD | UnD | UnD | UnD | UnD | UnD |
| | N2 | UnD | UnD | NA | UnD | UnD | UnD | UnD | UnD | UnD |
| | RNaseP | 35.0 | 37.1 | 36.5 | 34.4 | 34.4 | 34.5 | 33.9 | 33.9 | 34.4 |
| | RNaseP | 35.3 | 36.1 | NA | 34.4 | 34.6 | 34.1 | 33.8 | 33.8 | 34.2 |

RT-qPCR CTRL

| Control | NTC | POS |
|---|---|---|
| 24 hrs | UnD | 38.0 |
| 72 hrs | UnD | UnD [symbol] |
| 24 hrs | UnD | 38.0 |
| 72 hrs | UnD | UnD [symbol] |
| 24 hrs | UnD | UnD |
| 72 hrs | UnD | UnD |
| Day 7 | UnD | 34.0 |
| | UnD | 35.2 |
| | UnD | 34.3 |
| | UnD | 34.3 |
| | UnD | UnD |
| | UnD | UnD |

| | |
|---|---|
| UnD [symbol] | RNA Stock degradation |

[symbol]

indicates data missing or illegible when filed

TABLE 5
CT value comparison of contrived clinical oropharyngeal samples stored in Formulation D, Formulation C or DNA/RNA
Shield ™ sample collection kits (Zymo Research and Pangea Laboratory).

| Swab Collection kit | | | Zymo/Pangea | | | | Formulation D | | | | Formulation C | | | |
|---|---|---|---|---|---|---|---|---|---|---|---|---|---|---|
| Extraction Methods | | | | | QiaAmp | | | | QiaAmp | | | | QiaAmp | |
| Swab | | | Magmax/200 ul | | Mini/140 ul | | Magmax/200 ul | | Mini/140 ul | | Magmax/200 ul | | Mini/140 ul | |
| Donor | RNA | | 24 | 1 | 24 | 6 | 24 | 1 | 24 | 6 | 24 | 1 | 24 | 6 |
| ID | spike | Target | Hrs | wk | Hrs | Days | Hrs | wk | Hrs | Days | Hrs | wk | Hrs | Days |
| A | 0 | N1 | UnD | UnD | UnD | UnD | UnD | UnD | UnD | UnD | UnD | UnD | UnD | UnD |
| | cps/Swab A | N1 | UnD | UnD | UnD | UnD | UnD | UnD | UnD | UnD | UnD | UnD | UnD | UnD |
| | | N2 | UnD | UnD | UnD | UnD | UnD | UnD | UnD | UnD | UnD | UnD | UnD | UnD |
| | | N2 | UnD | UnD | UnD | UnD | UnD | UnD | UnD | UnD | UnD | UnD | UnD | UnD |
| | | RNaseP | 30.0 | 29.2 | 32.7 | 33.3 | 30.3 | 30.3 | 31.7 | 31.1 | 29.6 | 30.0 | 33.2 | 32.0 |
| | | RNaseP | 28.6 | 29.7 | 33.3 | 32.8 | 30.2 | 29.2 | 31.7 | 31.1 | 29.3 | 28.4 | 32.8 | 32.2 |
| | 3 | N1 | 35.3 | 36.6 | 37.5 | UnD | 35.1 | 36.9 | 38.2 | UnD | 36.2 | 35.3 | 36.9 | 38.2 |
| | cps/Swab A | N1 | 37.6 | 36.1 | 38.2 | 36.8 | 34.8 | 34.9 | 37.1 | 35.3 | 34.3 | 35.3 | UnD | 36.7 |
| | | N2 | 35.5 | 36.1 | 37.3 | 37.6 | 37.2 | 36.0 | 37.2 | 36.5 | 36.0 | 36.4 | UnD | UnD |
| | | N2 | UnD | 36.1 | UnD | 38.2 | 36.8 | 35.9 | 38.0 | UnD | 35.9 | 36.5 | 40.0 | UnD |
| | | RNaseP | 27.2 | 28.1 | 33.7 | 33.6 | 30.0 | 30.6 | 31.7 | 30.8 | 29.7 | 28.9 | 32.5 | 32.2 |
| | | RNaseP | 27.5 | 28.6 | 33.7 | 32.7 | 30.6 | 28.5 | 32.3 | 30.8 | 29.8 | 28.2 | 32.2 | 32.4 |
| B | 0 | N1 | UnD | UnD | UnD | UnD | UnD | UnD | UnD | UnD | — | — | UnD | UnD |
| | cps/Swab B | N1 | UnD | UnD | UnD | UnD | UnD | UnD | UnD | UnD | — | — | UnD | UnD |
| | | N2 | UnD | UnD | UnD | UnD | UnD | UnD | UnD | UnD | — | — | UnD | UnD |
| | | N2 | UnD | UnD | UnD | UnD | UnD | UnD | UnD | UnD | — | — | UnD | UnD |
| | | RNaseP | 28.1 | 28.4 | 30.8 | 30.7 | 27.1 | 27.0 | 30.9 | 30.4 | — | — | 31.6 | 31.4 |
| | | RNaseP | 28.3 | 28.4 | 30.9 | 30.7 | 27.4 | 27.2 | 30.8 | 30.9 | — | — | 31.9 | 31.2 |
| | 3 | N1 | 37.6 | 37.8 | 36.6 | UnD | 34.2 | 36.1 | UnD | 35.6 | — | — | UnD | 37.9 |
| | cps/Swab B | N1 | 36.1 | 37.2 | UnD | UnD | 35.2 | 34.9 | 37.2 | UnD | — | — | 37.4 | 36.7 |
| | | N2 | 36.4 | 36.3 | UnD | 37.7 | 35.4 | 35.6 | UnD | 36.8 | — | — | UnD | 39.4 |
| | | N2 | 37.9 | 37.0 | 38.8 | UnD | 36.6 | 35.9 | 38.6 | 38.0 | — | — | 39.2 | 39.0 |
| | | RNaseP | 27.1 | 27.6 | 30.5 | 29.8 | 28.5 | 28.2 | 30.9 | 30.4 | — | — | 32.8 | 31.8 |
| | | RNaseP | 27.1 | 27.8 | 30.3 | 29.8 | 28.5 | 27.8 | 30.9 | 31.1 | — | — | 32.4 | 31.9 |

| RT-qPCR | Magmax | | QiaAmp Mini | |
|---|---|---|---|---|
| Controls | 24 Hrs | 1 wk | 24 Hrs | 6 Days |
| NTC | UnD | UnD | UnD | UnD |
| | NA | UnD | UnD | UnD |
| | UnD | UnD | UnD | UnD |
| | NA | UnD | UnD | UnD |
| | UnD | UnD | UnD | UnD |
| | NA | UnD | UnD | UnD |
| POS | 36.8 | 36.0 | 38.5 | 32.3 |
| | NA | 36.0 | 37.4 | 32.2 |
| | 42. [symbol] | 36.0 | 37.9 | 33.6 |
| | NA | 35.0 | UnD | 33.7 |
| | UnD | UnD | UnD | UnD |
| | NA | UnD | UnD | UnD |

[symbol]

indicates data missing or illegible when filed

Notably, Formulation D samples showed less detection dropout among contrived positive samples, which may suggest better sensitivity for downstream assays. In addition, the detection sensitivity of Formulation D and C was verified at 1-3 copies/4, with included 3 copies/4, contrived samples.

### Example 5: Performance Comparison with Another Commercial Sample Transport Medium

The performance of another commercially available sample transport medium from Precision Medical Group (PMG) was compared with the performance of Formulation D. 1.0 mL of Formulation D or PMG sample transport medium was used to fill a 15 mL sterile conical tube to simulate sample transport tubes for all samples in the following experiment.

10 μL pooled healthy donor serum was added as an internal human specimen control (HSC) to each of the tubes containing 1.0 mL Formulation D or PMG transport medium. The tubes were mixed by pulse vortexing, spun, and stored at room temperature for 10 minutes. 9 μL 10,000 copies/μL SAR-CoV-2 RNA was diluted in 11 μL nuclease free water to achieve 4500 copies/μL stock solution. 5 μL of 4500 copies/μL solution was transferred to 10 μL nuclease free water to get 1500 copies/μL; then 5 μL of 1500 copies/μL solution was transferred to 10 μL nuclease free water to get 500 cps/μL. 2 μL of 4500 copies/μL, 1500 copies/μL, or 500 copies/μL SARS-CoV-2 RNA solution was added to each of the transport tubes containing 1.0 mL Formulation D or PMG transport medium and 10 μL HSC added to achieve 9 copies/μL, 3 copies/μL or 1 copy/μL SAR-CoV-2 RNA, respectively. 2 μL nuclease free water was used to replace RNA as negative controls. The tubes were mixed by pulse vortexing, spun, and stored at room temperature for 1 day, 2 days and 7 days for follow on nucleic acid extraction. RNA extraction and RT-qPCR was performed as described in Example 1.

Samples preserved in Formulation D showed positive detection from 1-3 copies/μL stored at room temperature up to 7 days (or even for 30 days, see Example 3). In comparison, SARS-CoV-2 RNA was detected in PMG preserved samples at 9 copies/μL up to 48 hours only. Samples with up to 9 copies/μL SARS-CoV-2 RNA failed detection on day 7. Therefore, these results demonstrate that Formulation D has superior properties in preserving pathogenic nucleic acids at room temperature for longer periods of time, as compared to commercially used sample transport media.

TABLE 6
CT value comparison of SARS-CoV-2 RNA samples stored in Formulation D and PMG sample transport medium

| Covid-19 | | | PMG | | | Formulation D | | | PCR Controls | | | |
|---|---|---|---|---|---|---|---|---|---|---|---|---|
| RNA | Extraction | | Day 1/ | Day 7/ | Day 2/ | Day 1/ | Day 7/ | Day 2/ | | | | POS |
| Spike | Rep | Target | MagMax | Magmax | QiaAmp | MagMax | Magmax | QiaAmp | Runs | Target | NTC | (5 cps/ul) |
| 9 cps/ul | Rep 1 | N1 | 34.7 | UnD | 36.5 | 34.3 | 33.6 | 34.1 | Day 1/ | N1 | UnD | 34.8 |
| | | N1 | 37.9 | UnD | UnD | 34.9 | 33.7 | 33.3 | MagMax | N1 | UnD | 35.8 |
| | | N2 | 37.6 | UnD | 37.8 | 34.3 | 35.0 | 33.5 | | N2 | UnD | 36.6 |
| | | N2 | UnD | UnD | 37.7 | 34.0 | 34.4 | 33.5 | | N2 | UnD | 36.9 |
| | | RNaseP | 35.4 | 34.3 | 33.7 | 33.9 | 33.7 | 34.6 | | RNaseP | UnD | UnD |
| | | RNaseP | 34.6 | 34.9 | 34.3 | 34.9 | 34.0 | 33.4 | | RNaseP | UnD | UnD |
| | Rep 2 | N1 | 36.3 | UnD | 36.2 | 35.0 | 33.4 | 34.3 | Day 2/ | N1 | UnD | 36.0 |
| | | N1 | 36.5 | UnD | 36.4 | 35.2 | 33.4 | 33.9 | QiaAmp | N1 | UnD | 37.3 |
| | | N2 | 36.2 | UnD | 36.9 | 34.5 | 34.1 | 35.0 | | N2 | UnD | 36.6 |
| | | N2 | 37.1 | UnD | 38.6 | 35.5 | 35.0 | 34.7 | | N2 | UnD | 36.4 |
| | | RNaseP | 34.7 | 35.4 | 33.8 | 33.8 | 33.9 | 33.2 | | RNaseP | UnD | UnD |
| | | RNaseP | 34.5 | 34.7 | 33.9 | 34.3 | 33.4 | 33.6 | | RNaseP | UnD | UnD |
| 3 cps/ul | Rep 1 | N1 | UnD | UnD | UnD | 35.4 | 38.1 | 35.8 | Day 7/ | N1 | UnD | 37.1 |
| | | N1 | 37.8 | UnD | 37.8 | 35.1 | 35.6 | 34.8 | MagMax | N1 | UnD | 36.4 |
| | | N2 | 38.8 | UnD | UnD | 36.7 | 36.4 | 35.6 | | N2 | UnD | 37.7 |
| | | N2 | UnD | UnD | 38.4 | 36.0 | 38.3 | 35.8 | | N2 | UnD | 36.1 |
| | | RNaseP | 35.4 | 33.9 | 33.5 | 33.8 | 34.3 | 34.4 | | RNaseP | UnD | UnD |
| | | RNaseP | 35.0 | 34.0 | 34.0 | 34.0 | 33.7 | 33.5 | | RNaseP | UnD | UnD |
| | Rep 2 | N1 | UnD | UnD | 37.6 | 36.0 | 36.0 | 34.9 | | | | |
| | | N1 | UnD | UnD | UnD | 35.2 | 36.1 | 35.5 | | | | |
| | | N2 | 38.9 | UnD | 38.6 | 37.1 | 36.7 | 36.0 | | | | |
| | | N2 | 39.1 | UnD | 37.5 | 38.9 | 39.0 | 35.7 | | | | |
| | | RNaseP | 34.3 | 34.5 | 33.3 | 33.2 | 34.0 | 34.0 | | | | |
| | | RNaseP | 36.0 | 35.3 | 34.3 | 34.1 | 34.1 | 34.4 | | | | |
| 1 cps/ul | Rep 1 | N1 | UnD | UnD | UnD | UnD | 37.6 | 37.0 | | | | |
| | | N1 | UnD | UnD | UnD | 38.8 | UnD | 36.5 | | | | |
| | | N2 | UnD | UnD | UnD | 40.0 | 37.6 | 36.0 | | | | |
| | | N2 | UnD | UnD | UnD | 39.0 | UnD | 36.6 | | | | |
| | | RNaseP | 34.7 | 35.0 | 34.0 | 34.0 | 35.0 | 33.6 | | | | |
| | | RNaseP | 34.1 | 35.1 | 34.0 | 34.5 | 34.0 | 33.5 | | | | |
| | Rep 2 | N1 | UnD | UnD | UnD | 36.9 | 37.3 | 35.6 | | | | |
| | | N1 | UnD | UnD | 37.1 | 36.9 | 35.4 | 37.8 | | | | |
| | | N2 | UnD | UnD | 38.6 | UnD | 38.7 | 37.2 | | | | |
| | | N2 | 38.8 | UnD | UnD | 38.2 | 37.4 | 37.4 | | | | |
| | | RNaseP | 34.4 | 35.2 | 33.7 | 34.6 | 33.5 | 34.0 | | | | |
| | | RNaseP | 34.3 | 34.6 | 33.9 | 34.0 | 33.4 | 33.7 | | | | |
| HSC | Rep 1 | N1 | UnD | UnD | UnD | UnD | UnD | UnD | | | | |
| (0 cp) | | N1 | UnD | UnD | UnD | UnD | UnD | UnD | | | | |
| | | N2 | UnD | UnD | UnD | UnD | UnD | UnD | | | | |
| | | N2 | UnD | UnD | UnD | UnD | UnD | UnD | | | | |
| | | RNaseP | 34.5 | 34.8 | 33.8 | 34.1 | 33.9 | 34.1 | | | | |
| | | RNaseP | 34.5 | 34.2 | 34.1 | 34.0 | 34.0 | 33.8 | | | | |

### Example 6: Compatibility with Multiple Nucleic Acid Isolation Platforms

MagMAX™ nucleic acid isolation kit (Thermo Fisher) and reagents use magnetic bead-based technology to purify high-quality nucleic acids from a range of research sample types both manually and through automation. In order to test the compatibility of Formulation D with a broad range of nucleic acid extraction platforms, we opted for QIAamp DSP Viral RNA Mini Kit (Qiagen) as representative for the widely used silica spin column-based RNA extraction methods.

The evaluation was conducted with contrived clinical samples. Oropharyngeal (OP) swab samples were collected from two healthy volunteers following CDC recommended sample collection protocols. All samples were collected in tubes containing 1 mL Formulation D, Formulation C, or DNA/RNA Shield™ (Zymo Research). 2 μL 10,000 copies/μL SAR-CoV-2 RNA was diluted in 18 μL Nuclease free water to achieve 1000 copies/μL RNA. 3 of 1000 copies/μL SARS-CoV-2 RNA was added to each of above OP swab samples as contrived positive clinical samples. 3 μL nuclease free water was added to replace RNA as a contrived negative clinical sample. All samples were stored at room temperature for 1 day and 7 days for follow on RNA extraction. RNA extraction and RT qPCR was performed as described in Example 1.

Comparison of CT values of contrived clinical samples stored in Formulation D and DNA/RNA Shield™ (Zymo Research) after RNA extraction with QIAamp DSP Viral RNA Mini Kit (Qiagen) or a MagMAX™ nucleic acid isolation kit (Thermo Fisher) is shown in Table 7.

TABLE 7
CT values of Formulation D or Zymo transport media preserved samples extracted by QIAamp DSP Viral RNA Mini Kit (Qiagen)

| Swab Collection kit | | | Zymo/Pangea | | | | Formulation D | | | | Formulation C | | | |
|---|---|---|---|---|---|---|---|---|---|---|---|---|---|---|
| Extraction Methods | | | | | QiaAmp | | | | QiaAmp | | | | QiaAmp | |
| Swab | | | Magmax/200 ul | | Mini/140 ul | | Magmax/200 ul | | Mini/140 ul | | Magmax/200 ul | | Mini/140 ul | |
| Donor | RNA | | 24 | 1 | 24 | 6 | 24 | 1 | 24 | 6 | 24 | 1 | 24 | 6 |
| ID | spike | Target | Hrs | wk | Hrs | Days | Hrs | wk | Hrs | Days | Hrs | wk | Hrs | Days |
| A | 0 | N1 | UnD | UnD | UnD | UnD | UnD | UnD | UnD | UnD | UnD | UnD | UnD | UnD |
| | cps/Swab A | N1 | UnD | UnD | UnD | UnD | UnD | UnD | UnD | UnD | UnD | UnD | UnD | UnD |
| | | N2 | UnD | UnD | UnD | UnD | UnD | UnD | UnD | UnD | UnD | UnD | UnD | UnD |
| | | N2 | UnD | UnD | UnD | UnD | UnD | UnD | UnD | UnD | UnD | UnD | UnD | UnD |
| | | RNaseP | 30.0 | 29.2 | 32.7 | 33.3 | 30.3 | 30.3 | 31.7 | 31.1 | 29.6 | 30.0 | 33.2 | 32.0 |
| | | RNaseP | 28.6 | 29.7 | 33.3 | 32.8 | 30.2 | 29.2 | 31.7 | 31.1 | 29.3 | 28.4 | 32.8 | 32.2 |
| | 3 | N1 | 35.3 | 36.6 | 37.5 | UnD | 35.1 | 36.9 | 38.2 | UnD | 36.2 | 35.3 | 36.9 | 38.2 |
| | cps/Swab A | N1 | 37.6 | 36.1 | 38.2 | 36.8 | 34.8 | 34.9 | 37.1 | 35.3 | 34.3 | 35.3 | UnD | 36.7 |
| | | N2 | 35.5 | 36.1 | 37.3 | 37.6 | 37.2 | 36.0 | 37.2 | 36.5 | 36.0 | 36.4 | UnD | UnD |
| | | N2 | UnD | 36.1 | UnD | 38.2 | 36.8 | 35.9 | 38.0 | UnD | 35.9 | 36.5 | 40.0 | UnD |
| | | RNaseP | 27.2 | 28.1 | 33.7 | 33.6 | 30.0 | 30.6 | 31.7 | 30.8 | 29.7 | 28.9 | 32.5 | 32.2 |
| | | RNaseP | 27.5 | 28.6 | 33.7 | 32.7 | 30.6 | 28.5 | 32.3 | 30.8 | 29.8 | 28.2 | 32.2 | 32.4 |
| B | 0 | N1 | UnD | UnD | UnD | UnD | UnD | UnD | UnD | UnD | — | — | UnD | UnD |
| | cps/Swab B | N1 | UnD | UnD | UnD | UnD | UnD | UnD | UnD | UnD | — | — | UnD | UnD |
| | | N2 | UnD | UnD | UnD | UnD | UnD | UnD | UnD | UnD | — | — | UnD | UnD |
| | | N2 | UnD | UnD | UnD | UnD | UnD | UnD | UnD | UnD | — | — | UnD | UnD |
| | | RNaseP | 28.1 | 28.4 | 30.8 | 30.7 | 27.1 | 27.0 | 30.9 | 30.4 | — | — | 31.6 | 31.4 |
| | | RNaseP | 28.3 | 28.4 | 30.9 | 30.7 | 27.4 | 27.2 | 30.8 | 30.9 | — | — | 31.9 | 31.2 |
| | 3 | N1 | 37.6 | 37.8 | 36.6 | UnD | 34.2 | 36.1 | UnD | 35.6 | — | — | UnD | 37.9 |
| | cps/Swab B | N1 | 36.1 | 37.2 | UnD | UnD | 35.2 | 34.9 | 37.2 | UnD | — | — | 37.4 | 36.7 |
| | | N2 | 36.4 | 36.3 | UnD | 37.7 | 35.4 | 35.6 | UnD | 36.8 | — | — | UnD | 39.4 |
| | | N2 | 37.9 | 37.0 | 38.8 | UnD | 36.6 | 35.9 | 38.6 | 38.0 | — | — | 39.2 | 39.0 |
| | | RNaseP | 27.1 | 27.6 | 30.5 | 29.8 | 28.5 | 28.2 | 30.9 | 30.4 | — | — | 32.8 | 31.8 |
| | | RNaseP | 27.1 | 27.8 | 30.3 | 29.8 | 28.5 | 27.8 | 30.9 | 31.1 | — | — | 32.4 | 31.9 |

| RT-qPCR | Magmax | | QiaAmp Mini | |
|---|---|---|---|---|
| Controls | 24 Hrs | 1 wk | 24 Hrs | 6 Days |
| NTC | UnD | UnD | UnD | UnD |
| | NA | UnD | UnD | UnD |
| | UnD | UnD | UnD | UnD |
| | NA | UnD | UnD | UnD |
| | UnD | UnD | UnD | UnD |
| | NA | UnD | UnD | UnD |
| POS | 36.8 | 36.0 | 38.5 | 32.3 |
| | NA | 36.0 | 37.4 | 32.2 |
| | 42.6 | 36.0 | 37.9 | 33.6 |
| | NA | 35.0 | UnD | 33.7 |
| | UnD | UnD | UnD | UnD |
| | NA | UnD | UnD | UnD |

Contrived clinical samples stored in Formulation D show positive detection after RNA extraction with QIAamp DSP Viral RNA Mini Kit (Qiagen) (Table 8). Since the sample input volume in standard protocols uses 140 μL instead of 200 μL as in the MagMAX™ workflow, the decreased sensitivity in detection is expected and can be compensated for by adjusting input or elution volumes when needed. QIAamp DSP Viral RNA Mini Kit (Qiagen) mini kits represent the silica spin column-based RNA extraction methods broadly used in US laboratories. Thus these results demonstrate that Formulation D is compatible with not only magnetic beads-based platforms such as MagMAX™, but also silica spin column-based RNA extraction methods. Since both of these methods are adaptable to automation, the feasibility of integrating Formulation D in an automatic workflow will be tested.

### Example 7: Formulation D Inactivates SARS-CoV-2 after Contact

To minimize the exposure of personnel to infectious viruses during sample handling and testing, Formulation D is designed to inactivate viruses such as SARS-CoV-2 upon contact. The following study was performed to evaluate the viricidal properties of Formulation D.

As used herein, TCID50 (Median Tissue Culture Infectious Dose) signifies the concentration at which 50% of the cells are infected when a test tube or well plate upon which cells have been cultured is inoculated with a diluted solution of the viral fluid. The TCID50/mL of CoV229E was determined following exposure to Formulation D for 2 hours at room temperature. One milliliter (1 mL) of CoV229E (neat) was incubated at room temperature with 1 mL of assay media, or Formulation D (neat) for 2 hours. An additional sample that includes 1 mL of Formulation D and 1 mL of assay media was also evaluated in parallel. Following the incubation, 1 mL of each mixture and 10 mL of DPBS were added to individual filter tubes (30,000 MW cutoff—Amicon Ultra 15, cat #UFC903008). The tubes were centrifuged at 4000×g at 4° C. for 30 minutes. This process was repeated two additional times except that the volume remaining in the filter apparatus was transferred to a new filter rather than 1 mL. A virus stock sample that was incubated with assay media for 2 hours but did not undergo centrifugation and filtering was also evaluated for the titer in parallel. Following the last centrifugation, an additional 2 mL of assay media was added to the tubes that were centrifuged and all samples were further diluted in serial 1:10 increments.

200 μL of each dilution was plated in quadruplicate wells of cells in a 96-well plate. The cultures were incubated for six (6) days. Following the incubation, the cells were stained with XTT to determine the TCID50/mL. XTT is a tetrazolium derivative that is turned into a water-soluble orange product after reduction by mitochondrial enzymes that are only present in metabolically active live cells. The Formulation D-treated cells only (no virus addition) were also stained with XTT to confirm no cytotoxicity.

TC50 values for the test materials were derived by measuring the reduction of XTT. XTT in metabolically active cells is metabolized by the mitochondrial enzyme NADPH oxidase to a soluble formazan product. XTT solution was prepared daily as a stock of 1 mg/mL in DMEM without additives. Phenazine methosulfate (PMS) solution was prepared at 0.15 mg/mL in DPBS and stored in the dark at −20° C. XTT/PMS stock was prepared immediately before use by adding 40 μL of PMS per mL of XTT solution. 50 μL of XTT/PMS was added to each well of the plate and the plate incubated for 4 hours at 37° C. The 4-hour incubation has been empirically determined to be within the linear response range for XTT dye reduction with the indicated numbers of cells for each assay. The plates were sealed and inverted several times to mix the soluble formazan product and the plate was read at 450 nm (650 nm reference wavelength) with a Molecular Devices SpectraMax Plus 384 96 well plate format spectrophotometer.

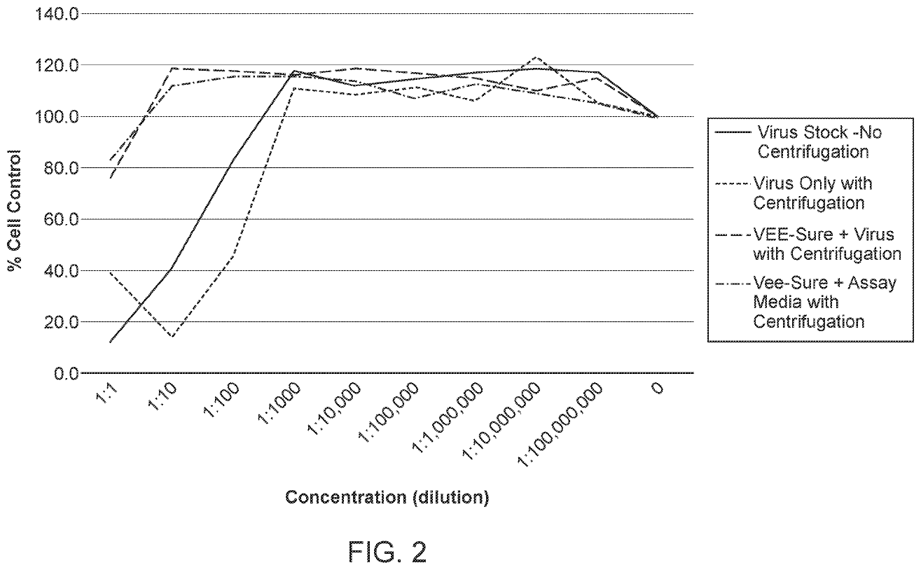

Formulation D was evaluated for viricidal activity to CoV229E following a two-hour exposure at room temperature. To minimize the inference of the toxicity of Formulation D to the cells, each sample was passed through a filtration spin column with a MW cut-off of 30,000 following the incubation. This filtration step allows the sample to flow through and the virus to remain thus removing the Formulation D after contact. The virus stock that did not undergo filtration following the incubation had a 50% titer of 2.634 and a TCID50/mL of 2153. The virus control that underwent filtration had a 50% titer of 1.801 and a TCID50/mL of 316. The Formulation D-treated virus sample and Formulation D-treated media had 50% titers of <0.301. A TCID50/mL could not be calculated. Data are presented in Table 8 and raw data are included in FIG. 2.

TABLE 8
Effect of Formulation D on the Infectivity
of CoV229E Following a 2 Hour Exposure

| Sample | 50% Titer | TCID₅₀/mL |
|---|---|---|
| Virus Stock - No Centrifugation | 2.634 | 2153 |
| Virus Only with Centrifugation | 1.801 | 316 |
| VEE-Sure + Virus with Centrifugation | <0.301 | *No value |
| VEE-Sure + Assay Media with Centrifugation | <0.301 | *No Value |

*No value—a value cannot be determined due to a titer that is “less than” a value.

The results of this experiment show that exposure of CoV229E with Formulation D (VEE-Sure) for 2 hours at room temperature reduced the infectivity to an undetectable level by the XTT assay. Therefore, CoV229E is inactivated upon contact with Formulation D.

Overall, the studies described above show that the pathogen transport media formulations disclosed herein are able to preserve the nucleic acids in a biological sample for long periods of time at room temperature, are compatible with multiple nucleic acid extraction methods, and can inactivate pathogens upon contact. Therefore, the pathogen transport media formulations disclosed herein have the superior properties that can revolutionize the collection, transport, storage and downstream processing of biological samples containing or suspected to contain pathogen, such as SARS-CoV-2.

### Numbered Embodiments

Embodiment 1. A formulation, comprising a sodium salt of a detergent, a metal chelating agent and a buffer, wherein the concentration of the detergent is in the range of about 0.1% to about 3%, wherein the concentration of the metal chelating agent is in the range of about 0.5 mM to about 1.5 mM, and wherein the concentration of the buffer is in the range of about 20 mM to about 40 mM.

Embodiment 2. The formulation of embodiment 1, wherein contact of the formulation with a biological sample results in one or more of the following: (a) inactivation of a nuclease in the biological sample, (b) inactivation of a pathogenic microbe in the biological sample, (c) disruption of an association of a protein with a nucleic acid in the biological sample, and (d) reduction or prevention of degradation of a nucleic acid in the biological sample, wherein the biological sample is derived from a subject, or the environment.

Embodiment 3. A formulation, comprising the following components: a sodium salt of a detergent, a metal chelating agent and a buffer, wherein contact of the formulation with a biological sample results in one or more of the following: (a) inactivation of a nuclease in the biological sample, (b) inactivation of a pathogenic microbe in the biological sample, (c) disruption of an association of a protein with a nucleic acid in the biological sample, and (d) reduction or prevention of degradation of a nucleic acid in the biological sample, wherein the biological sample is derived from a subject.

Embodiment 4. The formulation of any one of embodiments 1-3, wherein the concentration of the detergent is in the range of about 1% to about 3%.

Embodiment 5. The formulation of embodiment 4, wherein the concentration of the detergent is about 2%.

Embodiment 6. The formulation of embodiment 4, wherein the concentration of the detergent is about 3%.

Embodiment 7. The formulation of any one of embodiments 1-6, wherein the detergent is selected from the group consisting of sodium lauryl sulfate, sodium lauryl sarcosine, sodium octyl sulfate, sodium dihexadecyl phosphate, sodium taurodeoxycholate, sodium taurocholate, sodium glycocholate, sodium deoxycholate, sodium cholate, and sodium alkylbenzene sulfonate.

Embodiment 8. The formulation of embodiment 7, wherein the detergent is sodium lauryl sulfate.

Embodiment 9. The formulation of any one of embodiments 1-8, wherein the metal chelating agent is selected from the group consisting of ethylene glycol tetraacetic acid (EGTA), hydroxyl ethylethylenediaminetriacetic acid, di ethyl ene tri amine penta acetic acid, N,N-bis(carboxymethyl)glycine, ethylene diamine tetraacetic acid (EDTA), citrate anhydrous, sodium citrate, calcium citrate, ammonium citrate, ammonium bicitrate, citric acid, diammonium citrate, ferric ammonium citrate, and lithium citrate.

Embodiment 10. The formulation of embodiment 9, wherein the metal chelating agent is EDTA, EGTA, or a combination thereof.

Embodiment 11. The formulation of any one of embodiments 1-10, wherein the concentration of the metal chelating agent is about 1 mM.

Embodiment 12. The formulation of any one of embodiments 1-11, wherein the buffer comprises tris(hydroxymethyl)aminomethane, citrate, 2-(N-morpholino)ethanesulfonic acid, N,N-Bis(2-hydroxyethyl)-2-aminoethanesulfonic acid, 1,3-bis(tris(hydroxymethyl)methyl amino)propane, 4-(2-hydroxyethyl)-1-piperazine ethanesulfonic acid, 3-(N-morpholino) propanesulfonic acid, bicarbonate, or phosphate.

Embodiment 13. The formulation of any one of embodiments 1-12, wherein the buffer comprises a phosphate buffer.

Embodiment 14. The formulation of any one of embodiments 1-13, wherein the concentration of the buffer is about 30 mM.

Embodiment 15. The formulation of any one of embodiments 1-14, wherein the formulation has a pH in the range of about 6.5 to about 7.5.

Embodiment 16. The formulation of embodiment 15, wherein the formulation has a pH of about 6.7.

Embodiment 17. The formulation of any one of embodiments 1-16, wherein the formulation does not comprise a chaotropic agent.

Embodiment 18. The formulation of embodiment 17, wherein the chaotropic agent is selected from the group consisting of n-butanol, ethanol, guanidine isocyanate, guanidine hydrochloride, guanidinium thiocyanate, guanidinium chloride, lithium perchlorate, lithium acetate, magnesium chloride, phenol, 2-propanol, thiourea, and urea.

Embodiment 19. The formulation of embodiment 18, wherein the chaotropic agent is guanidinium thiocyanate.

Embodiment 20. The formulation of any one of embodiments 1-19, wherein the formulation does not comprise a reducing agent.

Embodiment 21. The formulation of embodiment 20, wherein the reducing agent is selected from the group consisting of 2-mercaptoethanol, tris(2-carboxyethyl)phosphine, dithiothreitol, dimethylsulfoxide, and tris(2-carboxyethyl)phosphine.

Embodiment 22. The formulation of any one of embodiments 1-21, wherein bringing the formulation in contact with bleach does not produce a hazardous compound.

Embodiment 23. The formulation of embodiment 22, wherein the hazardous compound is hydrogen cyanide.

Embodiment 24. The formulation of any one of embodiments 2-23, wherein the biological sample comprises a nasal sample, an oral sample, a saliva sample, a urine sample, a stool sample, a bronchoalveolar lavage sample, a nasopharyngeal aspirate sample or a blood sample.

Embodiment 25. The formulation of embodiment 24, wherein the biological sample comprises a nasal sample, an oral sample, a bronchoalveolar lavage sample, a nasopharyngeal aspirate sample or a saliva sample.

Embodiment 26. The formulation of embodiment 24, wherein the biological sample is obtained from a nasopharyngeal swab, an oropharyngeal swab, or a combination thereof.

Embodiment 27. The formulation of any one of embodiments 2-26, wherein contact of the formulation with the biological sample results in inactivation of a nuclease in the biological sample.

Embodiment 28. The formulation of embodiment 27, wherein the nuclease is an RNase.

Embodiment 29. The formulation of any one of embodiments 2-28, wherein the biological sample comprises, or is suspected to comprise a nucleic acid derived from a pathogenic microbe.

Embodiment 30. The formulation of any one of embodiments 2-29, wherein the biological sample comprises, or is suspected to comprise a pathogenic microbe.

Embodiment 31. The formulation of any one of embodiments 2-30, wherein the subject has or is at a risk of developing a disease associated with, correlated with, or caused by a pathogenic microbe.

Embodiment 32. The formulation of any one of embodiments 2-31, wherein contact of the formulation with the biological sample results in inactivation of a pathogenic microbe in the biological sample.

Embodiment 33. The formulation of any one of embodiments 29-32, wherein the pathogenic microbe is capable of causing disease in a healthy subject.

Embodiment 34. The formulation of embodiment 33, wherein the inactivated pathogenic microbe is incapable of causing disease in the healthy subject.

Embodiment 35. The formulation of any one of embodiments 29-34, wherein the pathogenic microbe is a bacterium, fungus, virus, or parasite.

Embodiment 36. The formulation of embodiment 35, wherein the pathogenic microbe is a virus.

Embodiment 37. The formulation of embodiment 36, wherein the virus is selected from the group consisting of SARS-CoV-2, SARS-CoV-1, MERS-CoV, chikungunya virus, African Swine Fever virus, Dengue virus, Zika virus, Influenza virus A, Influenza virus B, Influenza virus C, Human Immunodeficiency Virus (HIV), Ebola virus, Hepatitis virus A, Hepatitis virus B, Hepatitis virus C, Hepatitis virus D, and Hepatitis virus E, herpes simplex virus type 1 (HSV-1), herpes simplex virus type 2 (HSV-2) and Human Papillomavirus.

Embodiment 38. The formulation of embodiment 37, wherein the virus is SARS-CoV2.

Embodiment 39. The formulation of any one of embodiments 2-38, wherein contact of the formulation with the biological sample results in disruption of an association of a protein with a nucleic acid in the biological sample.

Embodiment 40. The formulation of embodiment 39, wherein the association of the protein with the nucleic acid comprises a physical binding of the protein with the nucleic acid.

Embodiment 41. The formulation of any one of embodiments 2-40, wherein contact of the formulation with a biological sample results in reduction or prevention of degradation of a nucleic acid in the biological sample.

Embodiment 42. The formulation of embodiment 41, wherein degradation of the nucleic acid is reduced or prevented for at least about 1 day.

Embodiment 43. The formulation of embodiment 41 or embodiment 42, wherein degradation of the nucleic acid is reduced or prevented for at least about 1 day at room temperature.

Embodiment 44. The formulation of any one of embodiments 41-43, wherein degradation of the nucleic acid is reduced or prevented for at least about 3 days at room temperature.

Embodiment 45. The formulation of any one of embodiments 41-44, wherein degradation of the nucleic acid is reduced or prevented for at least about 1 month at room temperature.

Embodiment 46. The formulation of any one of embodiments 39-45, wherein the nucleic acid is derived from a pathogenic microbe.

Embodiment 47. The formulation of any one of embodiments 39-46, wherein the nucleic acid is an RNA, a DNA, or a combination thereof.

Embodiment 48. The formulation of embodiment 47, wherein the nucleic acid is an RNA.

Embodiment 49. The formulation of embodiment 48, wherein the RNA is a SARS-CoV-2 genomic RNA.

Embodiment 50. A formulation, comprising: (a) about 3% sodium lauryl sulfate; (b) about 1 mM each of the EDTA and EGTA; and (c) about 30 mM of phosphate buffer; wherein the formulation has a pH of about 6.7, and wherein the formulation does not comprise guanidinium thiocyanate.

Embodiment 51. A container comprising the formulation of any one of embodiments 1-50.

Embodiment 52. A sample collection system, comprising: a collection device or a collection vessel; and the formulation of any one of embodiments 1-50.

Embodiment 53. A method of preparing a composition, comprising (a) collecting a biological sample from a subject, and (b) bringing the biological sample in contact with the formulation of any one of embodiments 1-50 to form the composition.

Embodiment 54. A method of transporting a biological sample derived from a subject, comprising (a) collecting the biological sample from the subject; (b) bringing the biological sample in contact with the formulation of any one of embodiments 1-50 to form a composition; and (c) transporting the composition.

Embodiment 55. The method of embodiment 53 or embodiment 54, wherein step (b) results in one or more of the following: (a) inactivation of a nuclease in the biological sample, (b) inactivation of a pathogenic microbe in the biological sample, (c) disruption of an association of a protein with a nucleic acid in the biological sample, and (d) reduction or prevention of degradation of a nucleic acid in the biological sample.

Embodiment 56. A method of inactivating a pathogenic microbe in a biological sample derived from a subject, comprising bringing the biological sample in contact with the formulation of any one of embodiments 1-50 to form a composition.

Embodiment 57. The method of embodiment 56, wherein the pathogenic microbe is capable of causing disease in a healthy subject.

Embodiment 58. The method of embodiment 57, wherein the inactivated pathogenic microbe is incapable of causing disease in the healthy subject.

Embodiment 59. The method of any one of embodiments 56-58, wherein the pathogenic microbe is a bacterium, fungus, virus, or parasite.

Embodiment 60. The method of embodiment 59, wherein the pathogenic microbe is a virus.

Embodiment 61. The method of embodiment 60, wherein the virus is selected from the group consisting of SARS-CoV-2, SARS-CoV-1, MERS-CoV, chikungunya virus, African Swine Fever virus, Dengue virus, Zika virus, Influenza virus A, Influenza virus B, Influenza virus C, Human Immunodeficiency Virus (HIV), Ebola virus, Hepatitis virus A, Hepatitis virus B, Hepatitis virus C, Hepatitis virus D, and Hepatitis virus E, herpes simplex virus type 1 (HSV-1), herpes simplex virus type 2 (HSV-2) and Human Papillomavirus.

Embodiment 62. The method of embodiment 61, wherein the virus is SARS-CoV2.

Embodiment 63. A method of detecting a nucleic acid in a biological sample, comprising (a) collecting the biological sample from a subject, (b) bringing the biological sample in contact with the formulation of any one of embodiments 1-50 to form a composition, and (c) detecting the nucleic acid in the composition.

Embodiment 64. A method of purifying a nucleic acid from a biological sample, comprising (a) collecting the biological sample from a subject, (b) bringing the biological sample in contact with the formulation of any one of embodiments 1-50 to form a composition, and (c) purifying the nucleic acid from the composition.

Embodiment 65. The method of embodiment 63, wherein the method comprises purifying the nucleic acid.

Embodiment 66. The method of embodiment 64 or embodiment 65, wherein purifying the nucleic acid comprises Boom extraction.

Embodiment 67. The method of embodiment 66, wherein Boom extraction comprises bringing the nucleic acid in contact with silica.

Embodiment 68. The method of embodiment 64 or embodiment 65, wherein purifying the nucleic acid comprises hybridization of the nucleic acid.

Embodiment 69. The method of any one of embodiments 63-68, wherein the nucleic acid is derived from a pathogenic microbe.

Embodiment 70. The method of any one of embodiments 63-69, wherein the nucleic acid is an RNA, a DNA, or a combination thereof.

Embodiment 71. The method of embodiment 70, wherein the nucleic acid is an RNA.

Embodiment 72. The method of embodiment 71, wherein the RNA is a SARS-CoV-2 genomic RNA.

Embodiment 73. The method of any one of embodiments 53-72, wherein the method comprises transporting and/or storing the composition at room temperature.

Embodiment 74. The method of any one of embodiments 53-73, wherein the method comprises transporting and/or storing the composition at room temperature for at least 1 day.

Embodiment 75. The method of any one of embodiments 53-74, wherein the method further comprises transporting and/or storing the composition at room temperature for at least 1 month.

Embodiment 76. The method of any one of embodiments 63-75, wherein the method comprises amplifying the nucleic acid.

Embodiment 77. The method of any one of embodiments 63-76, wherein the method comprises amplifying the nucleic acid after storing the composition at room temperature for at least 1 day.

Embodiment 78. The method of any one of embodiments 63-77, wherein the method comprises amplifying the nucleic acid after storing the composition at room temperature for at least 3 days.

Embodiment 79. The method of any one of embodiments 63-78, wherein the method comprises amplifying the nucleic acid after storing the composition at room temperature for at least 1 month.

Embodiment 80. A kit comprising the formulation of any one of embodiments 1-50 and/or the container of embodiment 51, and/or the sample collection system of embodiment 52.

1. A formulation for lysing cells in a biological sample and stabilizing the RNA contained in said cells, comprising a detergent, a metal chelating agent and a buffer, wherein the concentration of the detergent is in the range of about 0.1% to about 3%, wherein the concentration of the metal chelating agent is in the range of about 0.1 mM to about 5.0 mM, wherein the concentration of the buffer is in the range of about 20 mM to about 40 mM and wherein contact of the formulation with a biological sample results in one or more of the following: (a) inactivation of a nuclease in the biological sample, (b) inactivation of a pathogenic microbe in the biological sample, (c) disruption of an association of a protein with a nucleic acid in the biological sample, and (d) reduction or prevention of degradation of a nucleic acid in the biological sample, wherein the biological sample is derived from a subject.

2-6. (canceled)

7. The formulation according to claim 1, wherein the detergent is selected from the group consisting of sodium lauryl sulfate, sodium lauryl sarcosine, sodium octyl sulfate, sodium dihexadecyl phosphate, sodium taurodeoxycholate, sodium taurocholate, sodium glycocholate, sodium deoxycholate, sodium cholate, and sodium alkylbenzene sulfonate.

8. (canceled)

9. The formulation according to claim 1, wherein the metal chelating agent is selected from the group consisting of ethylene glycol tetraacetic acid (EGTA), hydroxyl ethylethylenediaminetriacetic acid, diethylene triamine penta acetic acid, N,N-bis(carboxymethyl)glycine, ethylene diamine tetraacetic acid (EDTA), citrate anhydrous, sodium citrate, calcium citrate, ammonium citrate, ammonium bicitrate, citric acid, diammonium citrate, ferric ammonium citrate, and lithium citrate.

10-11. (canceled)

12. The formulation according to claim 1, wherein the buffer comprises tris(hydroxymethyl)aminomethane, citrate, 2-(N-morpholino)ethanesulfonic acid, N,N-Bis(2-hydroxyethyl)-2-aminoethanesulfonic acid, 1,3-bis(tris(hydroxymethyl)methyl amino)propane, 4-(2-hydroxy ethyl)-1-piperazine ethanesulfonic acid, 3-(N-morpholino) propanesulfonic acid, bicarbonate, or phosphate.

13-14. (canceled)

15. The formulation according to claim 1, wherein the formulation has a pH in the range of about 6.5 to about 7.5.

16-23. (canceled)

24. The formulation according to claim 1, wherein the biological sample comprises a nasal sample, an oral sample, a saliva sample, a urine sample, a stool sample, a bronchoalveolar lavage sample, a nasopharyngeal aspirate sampled cerebrospinal fluid sample, a pleural effusion sample, a peritoneal effusion sample, or a blood sample.

25-27. (canceled)

28. The formulation according to claim 1, wherein the nuclease in the biological sample is an RNase.

29-34. (canceled)

35. The formulation according to claim 1, wherein the pathogenic microbe is a bacterium, fungus, virus, or parasite.

36. (canceled)

37. The formulation according to claim 35, wherein the virus is selected from the group consisting of SARS-CoV-2, SARS-CoV-1, MERS-CoV, chikungunya virus, African Swine Fever virus, Dengue virus, Zika virus, Influenza virus A, Influenza virus B, Influenza virus C, Human Immunodeficiency Virus (HIV), Ebola virus, Hepatitis virus A, Hepatitis virus B, Hepatitis virus C, Hepatitis virus D, and Hepatitis virus E, herpes simplex virus type 1 (HSV-1), herpes simplex virus type 2 (HSV-2) and Human Papillomavirus.

38-41. (canceled)

42. The formulation according to claim 1, wherein degradation of the nucleic acid is reduced or prevented for at least about 1 day, at least about 1 day at room temperature, at least 3 days at room temperature or at least about 1 month at room temperature.

43-45. (canceled)

46. The formulation according to claim 42, wherein the nucleic acid is derived from a pathogenic microbe.

47. The formulation according to claim 42, wherein the nucleic acid is an RNA, a DNA, or a combination thereof.

48-49. (canceled)

50. A formulation, comprising: (a) about 3% sodium lauryl sulfate; (b) about 1 mM each of the EDTA and EGTA; and (c) about 30 mM of phosphate buffer; wherein the formulation has a pH of about 6.7, and wherein the formulation does not comprise guanidinium thiocyanate.

51. (canceled)

52. A sample collection system, comprising: a collection device or a collection vessel; and the formulation according to claim 1.

53-63. (canceled)

64. A method of purifying a nucleic acid from a biological sample, comprising (a) collecting the biological sample from a subject, (b) bringing the biological sample in contact with the formulation according to claim 1 to form a composition, and (c) purifying the nucleic acid from the composition.

65-73. (canceled)

74. The method according to claim 64, wherein the method comprises transporting and/or storing the composition at room temperature for at least about 1 day, for at least about 1 day at room temperature, at least about three days at room temperature or at least about a month at room temperature.

75-80. (canceled)
